SEQUENCE LISTING (24 sequences)

SEQ ID NO: 1 - DNA, 19 bp, synthetic construct
  source 1..19: mol_type = other DNA
tttgcccggagaacaagag

SEQ ID NO: 2 - DNA, 21 bp, synthetic construct
  source 1..21: mol_type = other DNA
tttggagataggcagcttcag

SEQ ID NO: 3 - DNA, 20 bp, synthetic construct
  source 1..20: mol_type = other DNA
gactggcaatgagaggcaaa

SEQ ID NO: 4 - DNA, 21 bp, synthetic construct
  source 1..21: mol_type = other DNA
cgtaaactttgccgcctatga

SEQ ID NO: 5 - DNA, 26 bp, synthetic construct
  source 1..26: mol_type = other DNA
ggtaggttaagacatgaaggatttgc

SEQ ID NO: 6 - DNA, 21 bp, synthetic construct
  source 1..21: mol_type = other DNA
ctacatgaccctgtgaccttg

SEQ ID NO: 7 - DNA, 22 bp, synthetic construct
  source 1..22: mol_type = other DNA
gcgtatgtcaccatgtctagtt

SEQ ID NO: 8 - DNA, 20 bp, synthetic construct
  source 1..20: mol_type = other DNA
atgggaaggtgaaggtcgga

SEQ ID NO: 9 - DNA, 41 bp, synthetic construct
  source 1..41: mol_type = other DNA
gcacagtcaaggccgagaataggtcggtgtgaacggatttg

SEQ ID NO: 10 - DNA, 21 bp, synthetic construct
  source 1..21: mol_type = other DNA
agcccgaggaatcaacaatag

SEQ ID NO: 11 - DNA, 22 bp, synthetic construct
  source 1..22: mol_type = other DNA
tttggacttcttgtgccatttc

SEQ ID NO: 12 - DNA, 22 bp, synthetic construct
  source 1..22: mol_type = other DNA
aggtgtgaggaaagttcaagag

SEQ ID NO: 13 - DNA, 20 bp, synthetic construct
  source 1..20: mol_type = other DNA
tcttcccaccacgagtgtaa

SEQ ID NO: 14 - DNA, 24 bp, synthetic construct
  source 1..24: mol_type = other DNA
actcggtacttttggtcccactca

SEQ ID NO: 15 - DNA, 23 bp, synthetic construct
  source 1..23: mol_type = other DNA
gcttgagccaacttgaataccat

SEQ ID NO: 16 - DNA, 21 bp, synthetic construct
  source 1..21: mol_type = other DNA
ggcacaggatttgtgagtttg

SEQ ID NO: 17 - DNA, 22 bp, synthetic construct
  source 1..22: mol_type = other DNA
agcattccagactttccatctc

SEQ ID NO: 18 - DNA, 21 bp, synthetic construct
  source 1..21: mol_type = other DNA
cagcgtcaaaggtggaggagt

SEQ ID NO: 19 - DNA, 43 bp, synthetic construct
  source 1..43: mol_type = other DNA
gccttctccatggtggtgaatgtagaccatgtagttgaggtca

SEQ ID NO: 20 - DNA, 21 bp, synthetic construct
  source 1..21: mol_type = other DNA
actgggtagaaagggaagaga

SEQ ID NO: 21 - DNA, 20 bp, synthetic construct
  source 1..20: mol_type = other DNA
ggagagcaatgacgatgagg

SEQ ID NO: 22 - DNA, 22 bp, synthetic construct
  source 1..22: mol_type = other DNA
cctactcgaagacttacccagt

SEQ ID NO: 23 - DNA, 20 bp, synthetic construct
  source 1..20: mol_type = other DNA
gaacccggaagtcatagcag

SEQ ID NO: 24 - DNA, 21 bp, synthetic construct
  source 1..21: mol_type = other DNA
gcattggggtgaatgatagca

(19) United States
(12) Patent Application Publication
(10) Pub. No.: US 2023/0263861 A1
(43) Pub. Date: Aug. 24, 2023

(54) COMPOSITIONS AND METHODS FOR PROMOTING HAIR GROWTH

(71) Applicant: Duke University, Durham, NC (US)

(72) Inventors: Jennifer Yunyan Zhang, Durham, NC (US); Jessica Shannon, Durham, NC (US)

(21) Appl. No.: 18/173,358

(22) Filed: Feb. 23, 2023

Related U.S. Application Data
(60) Provisional application No. 63/312,867, filed on Feb. 23, 2022.

(51) Int. Cl. A61K 38/20; A61P 17/14; A61K 9/00

(52) U.S. Cl. CPC A61K 38/20; A61K 9/0021; A61P 17/14

(57) ABSTRACT

Provided herein are methods for promoting hair growth in a subject involving providing TSLP to the subject. Further provided herein are methods for preventing hair loss in a subject, including chemotherapy-induced hair loss, by inhibiting TSLP in the subject.

### STATEMENT REGARDING RELATED APPLICATIONS

This application claims priority to U.S. Provisional Pat. Application No. 63/312,867, filed Feb. 23, 2022, the entire contents of which are incorporated herein by reference for all purposes.

### STATEMENT REGARDING FEDERAL FUNDING

This invention was made with Government support under Federal Grant no. R01AI39207 awarded by the National Institute of Allergy and Infectious Diseases (NIH/NIAID). The Federal Government has certain rights to this invention.

### SEQUENCE LISTING

The text of the computer readable sequence listing filed herewith, titled “DUKE-41678-202_SQL”, created Feb. 23, 2023, having a file size of 22,225 bytes, is hereby incorporated by reference in its entirety.

### BACKGROUND

Hair follicles (HFs) are a defining feature of mammals and function as self-renewing miniature organs. Specifically, HFs harbor stem cells that control cyclic growth of hair follicles under homeostatic conditions and contribute to re-epithelialization of the interfollicular epidermis upon wounding. The HF microenvironment controls the activation state of hair follicle stem cells (HFSCs) and supports continuous renewal of hair by cycling through phases of anagen (active hair growth), catagen (regression), and telogen (resting). Loss of HFSC function results in the loss and/or inability to grow hair due to a variety of etiologies, such as chemotherapy and radiotherapy for cancer treatment, autoimmune-mediated destruction of HFSCs in alopecia areata, and stunted anagen cycles in androgenic alopecia (AGA). Symptoms of hair disorders are multidimensional that can negatively affect psychological health, psycho-social relationships, and other factors that impact quality of life. Accordingly, what is needed are compositions and methods of promoting hair growth.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the discovery by the inventors on the identification of TSLP as a potent inducer of hair growth in response to skin injury. The data shows that local delivery of exogenous TSLP promotes hair growth both in the presence and absence of skin injury. Using Lgr5CreER.Tslprfl/fl mice, it was demonstrated that TSLP acts through TSLPR on LGR5+ keratinocytes to promote expansion of TACs both during wound healing and normal tissue homeostasis. Further, it was found that TSLP increased expression of the cell cycle regulator cyclin D1 and the progenitor factor DDX6 in a TSLPR-dependent manner. The findings provided herein delineate TSLP as a novel and locally produced cytokine that directly stimulates hair follicle cell proliferation in the skin.

In some aspects, provided herein are methods of promoting hair growth in a subject. A method of promoting hair growth in a subject, comprising providing a composition containing a therapeutically effective amount of thymic stromal lymphopoietin (TSLP) to a subject in need thereof, thereby promoting hair growth in the subject. In some embodiments, composition is provided to the subject subcutaneously. In some embodiments, the therapeutically effective amount of TSLP is about 0.01 µg/kg to about 20 µg/kg. In some embodiments, the therapeutically effective amount of TSLP is about 0.025 µg/kg to about 10 µg/kg. In some embodiments, the therapeutically effective amount of TSLP is about 0.5 µg/kg to about 5 µg/kg. In some embodiments, the therapeutically effective amount of TSLP is about 1 µg/kg to about 3 µg/kg. In some embodiments, the subject has experienced a skin injury. In some embodiments, the composition is provided to the subject subcutaneously at an area of injured skin. In some embodiments, the subject has a hair disorder. In some embodiments, the hair disorder is alopecia.

In some embodiments, provided herein is a method of promoting hair growth in a subject afflicted with alopecia. In some embodiments, the method comprises providing a composition containing a therapeutically effective amount of thymic stromal lymphopoietin (TSLP) to the subject. In some embodiments, the composition is provided to the subject following a microneedling procedure performed on the subject for the treatment of alopecia. In some embodiments, the composition is administered subcutaneously at one or more microneedling sites. In some embodiments, the therapeutically effective amount of TSLP is about 0.01 µg/kg to about 20 µg/kg. In some embodiments, the therapeutically effective amount of TSLP is about 0.025 µg/kg to about 10 µg/kg. In some embodiments, the therapeutically effective amount of TSLP is about 0.5 µg/kg to about 5 µg/kg. In some embodiments, the therapeutically effective amount of TSLP is about 1 µg/kg to about 3 µg/kg.

In some aspects, provided herein are methods of preventing chemotherapy-induced hair loss in a subject. In some embodiments, methods of preventing chemotherapy-induced hair loss in a subject comprise providing a composition containing a therapeutically effective amount of an inhibitor of thymic stromal lymphopoietin (TSLP) to the subject before, during, and/or after chemotherapy, thereby preventing chemotherapy-induced hair loss in the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIGS. 1A-1H show that TSLP is produced in the skin in response to injury. FIG. 1A is a schematic illustration representing locus for human TSLP. FIG. 1B shows qPCR analysis of TSLP variants in human skin 24 hours after wounding; Data presented are from 4 independent experiments using 4 different human donors in technical duplicates. FIG. 1C shows immunostaining of human skin for TSLP total or long form TSLP (lfTSLP, red) and KRT15 (green). Images presented are representative from 3 independent experiments using 3 different human donors. Scale bar:100 µm. FIG. 1D shows qPCR analysis of lfTSLP and sfTSLP of human skin 24 hours after ex vivo wounding. Data from technical duplicates or triplicates of 4 experiments using 4 different human donors. FIG. 1E shows qPCR analysis of Tslp in RNA isolated from healing back skin wounds normalized to NW. Data from 3 independent experiments, 4 pooled wounds/animal, n=3 mice (NW, 4DPW, 30DPW) or n=6 mice (1DPW, 7DPW) in technical triplicates. FIG. 1F shows ELISA of TSLP in whole tissue lysates of skin wounds (2 independent experiments, n=2-3 mice per group). FIG. 1G shows immunostaining for TSLP (red) and ITGα6 (green) in non-wounded skin (top) or 7 DPW (bottom) skin from WT C57BL6 female mice. Scale bar: 20 µm. FIG. 1H shows immunostaining for TSLP (red) and CD45 (green) 7 DPW. All scale bars: 100 µm. p<0.01 **, p<0.001 ***, p<0.0001 ****. Error bars represent ± SEM. FC, fold change; NW, non-wounded; W, wounded; DPW, days post wounding. See also FIG. 7.

FIGS. 2A-2H show that TSLP is expressed throughout the hair cycle and accelerates onset of wound-induced hair growth in accordance with one embodiment of the present disclosure. FIG. 2A shows Tslp expression by qPCR. Data represent 10-31 mice per group from 4 independent experiments. Telogen’ denotes telogen following a complete hair cycle. FIG. 2B shows TSLP concentration in skin tissue by ELISA. Data represent 9-24 mice per group from 4 independent experiments. FIG. 2C shows immunostaining of skin samples collected from mice in anagen 8 days after depilation. TSLP (red), KRT6a (green), and nuclei (blue). Scale bars: 100 µm. FIG. 2D shows an exemplary experimental timeline indicating hair follicle stages and treatments of vehicle (0.01% BSA) or recombinant mouse TSLP (100 ng/wound) following 4-mm punch biopsy. FIG. 2E shows quantification of skin area that entered anagen; represents data from 3 experiments using n=8 mice per group. FIG. 2F shows immunostaining for Ki67, FIG. 2G shows Ki67 quantification, and FIG. 2H shows hair follicle length measurement from mouse skin 13 days after treatment with vehicle or TSLP. Data from 2 experiments using n=3 mice per group; 11-30 images per group were analyzed. Scale bars: 50 µm. *p<0.05, **p< 0.01, ***p< 0.001, and ****p< 0.0001.

FIGS. 3A-3K show TSLP expands CD34+ ITGα6lo transit amplifying cells (TAC). FIG. 3A shows an exemplary experimental timeline for hair growth analysis following s.c. TSLP (250 ng). FIG. 3B shows quantification of skin area that has entered anagen. Data from n=3 mice per group. FIG. 3C shows representative photos of mouse back skin after s.c. TSLP treatment. FIG. 3D shows experimental timeline for analysis of TSLP-driven cell proliferation. FIG. 3E shows representative flow cytometry plots of CD34+ cells. FIG. 3F shows ITGα6 expression pre-gated on CD34+. FIG. 3G shows quantification of CD34+ cells pre-gated on live, single cells. FIG. 3H shows quantification of total, live, EdU+ cells. FIG. 3I shows quantification of CD34+ ITGα6 subpopulations. FIG. 3J shows flow cytometry histogram overlay of CD34+ITGα6lo cells from TSLP and vehicle treated groups. FIG. 3K shows quantification of EdU+CD34+ITGα6lo. Graphs represent means of 2 experiments using 6-8 mice per group ± SEM. * p<0.05, ** p<0.01, ***p<0.001, ****p< 0.0001.

FIGS. 4A-4H show that murine epithelial cells express TSLPR. FIG. 4A shows representative flow cytometry plots pre-gated on live, single cells. FIG. 4B shows quantification of TSLPR+ relative cell abundance. FIG. 4C shows mean fluorescence intensity of surface expression of TSLPR on cells from hair follicle (HF) and skin from non-wounded tissue. Data presented represent averages from 4 experiments using n=7 mice (HF) and n=20 mice (skin) ± SEM. FIG. 4D is a schematic showing location of tissue section portrayed in FIG. 4E. FIG. 4 shows immunostaining of hair follicles neighboring 7-day old wound bed. TSLPR (red) and KRT6a (blue). Scale bars: 50 um. F. Flow cytometry for TSLPR+ cells after wounding. Data from 2 experiments using n=2-4 mice per group in technical duplicates. FIG. 4G shows immunostaining TSLPR (red) of 7-day old wound beds. Images 1-2 inserts were enlarged to show detail. Scale bars: 200 µm. FIG. 4H shows TSLPR immunostaining in non-wounded mouse back skin in telogen. Scale bars:50 µm. Keratin 6a (KRT6a, green) and nuclei (blue).

FIGS. 5A-5J show that TSLPR expression by LGR5+ HFSC promotes WHIG. FIGS. 5A-5G show quantification of TSLPR+ cells by flow cytometry from Lgr5CreER.Tslprfl/fl mice and Lgr5CreER .Tslprfl/+mice treated daily for 3 consecutive days with 4-hydroxytamoxifen (4OHT). FIG. 5A shows LGR5-eGFP+ cells pre-gated on total live cells. FIG. 5B shows TSLPR+ cells pre-gated on LGR5eGFP+ cells. FIG. 5C shows a flow plot and FIG. 5D shows quantification of TSLPR+LGR5+ cells, pre-gated on live, single cells. FIG. 5E shows a histogram for TSLPR expression from total LGR5eGFP+ cells. FIG. 5F shows a flow cytometry plot and FIG. 5G shows quantification of TSLPR+CD45+ cells, pre-gated on live, single cells. Data from 2 independent experiments using 5-7 mice/group. FIG. 5H shows an exemplary experimental timeline of quantification of skin that has entered anagen following wounding. FIG. 5I shows representative photos of mouse back skin after 4OHT treatment of Lgr5CreER.Tsfprfl/fl and Lgr5CreER.Tslprfl/+ mice. FIG. 5J shows quantification of anagen skin area of mice treated with or without TSLP. Graph represents averages of n=4-6 mice per group across 3 experiments ± SEM. p<0.05, ** p<0.01, ***p<0.001, ****p< 0.0001.

FIGS. 6A-6G show that TSLP promotes accumulation of progenitor factor DDX6. FIG. 6A and FIG. 6B show qPCR analysis of DDX6, KLF4, and FLG of RNA isolated from primary human keratinocytes treated with sfTSLP or lfTSLP for (FIG. 6A) 16 hours or (FIG. 6B) 24 hours. FIG. 6C shows immunostaining of DDX6 (red) in primary human keratinocytes stimulated with human TSLP (100 ng/mL) for 20 h. Scale bars: 20 µm. FIG. 6D shows quantification of DDX6. Graph represents average DDX6 intensity from n=17-28 images/group ± SEM; ****p< 0.0001. FIG. 6E shows a representative histogram of Ki67 staining of primary human keratinocytes stimulated with vehicle control, 3 nM lfTSLP or sfTSLP for 40 hours. Isotype IgG used for control. FIG. 6F and FIG. 6G show quantification of Ki67+ (FIG. 6F) cell abundance and (FIG. 6G) Ki67 MFI in primary human keratinocytes stimulated with 3 nM lfTSLP and sfTSLP for 24 hours. Data represent averages of 3 independent experiments using 2-3 different human donors in technical triplicates ± SEM. ** p<0.01, ***p<0.001, ****p< 0.0001. See also Table S1.

FIGS. 7A-7E show that human lfTSLP is expressed in glandular structures in the dermis. Related to FIG. 1. FIG. 7A shows immunostaining lfTSLP in lesional and non-lesional tissue samples collected from atopic dermatitis patients. Scale = 100 µm. FIG. 7B, FIG. 7C show representative immunostaining of human skin of TSLPₜₒₜₐₗ or long form TSLP (red) and CD34 or KRT77 (green) Data presented are from n=3 independent experiments using 3 different human donors. Scale bar = 100 µm. FIG. 7D shows qPCR results showing ΔΔC_{T} value for gene expression of each TSLP isoform from epidermis or dermis in response to 24h wound ex vivo. Values are normalized to NW epidermis for this tissue sample. Data presented are from n=4 independent experiments using 2 different human donors in technical duplicate. Error bars represent ± SEM. FIG. 7E is a schematic illustration representing cross section of human skin with sweat gland and hair follicle residing in dermis.

FIGS. 8A-8I show TSLP accelerates WIHG. Related to FIG. 2. FIG. 8A shows photos of mouse skin during active hair cycling showing changes in skin pigment in C57BL/6 mice. FIG. 8B shows qPCR of gene expression of Il7r and Crlf2 in whole skin in each hair follicle stage. Data presented are from n=2 experiments using n=27 mice (anagen I-IV); n=8 mice (anagen V); n=9 mice (anagen VI); n=3 mice (catagen); n=3 mice(telogen). FIG. 8C shows qPCR analysis of Tslp from wounded or non-wounded back skin (5dpw). Hair cycles of mice were synchronized, and wounds were induced during telogen or anagen (5 days after depilation) with timeline (right). Data presented are from n=1 experiment using n=3 mice per group. FIG. 8D shows representative photographs of healing mouse wounds (4-mm full thickness wounds) after treatment with TSLP (100 ng/wound) or vehicle. FIG. 8E, FIG. 8F show photographs of healing mouse wounds (FIG. 8E) and quantification (FIG. 8F) of total skin area that has entered anagen during WIHN assay (wound area=1.2 cm²) treated with mTslp (250 ng total) or vehicle (0.1%BSA) on the day of wounding. Data presented are from n=2 experiments using n=4-6 mice per group. FIG. 8G, FIG. 8H show photographs (FIG. 8G) and quantification of skin in anagen (FIG. 8H) from mice after WIHN assay (wound area=1.2 cm²) treated with TSLPR neutralizing antibody (1 ug total) or Rat IgG2A (1 ug) on the day of wounding and 4 days after wounding. Photographs are representative of 4 mice per group across 1 experiment on 23DPW and 32DPW. nAb, neutralizing TSLPR antibody. IgG, Immunoglobulin. control. FIG. 8I shows measurements of hair follicle at widest point (left) and epidermal thickness (right) from mouse skin 13 days after treatment with vehicle or TSLP Data presented are from n=2 experiments using n=3 mice per group and 11-30 unique sections of tissue were analyzed. Statistics determined using t-test where p<0.01 **, p<0.0001**** Error bars represent ± SEM.

FIGS. 9A-9E show epithelial Tslpr is required for timely entry of hair cycling and maintenance of normal epidermal thickness. FIG. 9A shows quantification of anagen entry by skin pigmentation intensity during second postnatal anagen cycle from Lgr5^{CreER}. Tslpr^{fl/fl} and littermate controls after 3 consecutive topical treatments of 4-OHT to back skin. FIG. 9B shows representative photos of mouse back skin after 4-OHT treatment. Representative of 9-12 mice per group across 3 independent experiments. FIG. 9C shows quantification of anagen entry by skin pigmentation during third anagen cycle. n=5-9 mice per group across 2 independent experiments. FIG. 9D shows representative photographs of mouse skin genotype after 4-OHT treatment. FIG. 9E shows representative images of H&E-stained control and HFSC^{Tslpr-/-} skin following 4-OHT treatment during telogen. FIG. 9F shows an exemplary experimental timeline and quantification of epidermal thickness after 4-OHT treatment during telogen. FIG. 9G shows representative images of H&E-stained control and HFSC^{Tslpr-/-} skin following 4-OHT treatment during anagen. FIG. 9H shows an exemplary experimental timeline and quantification of epidermal thickness after 4-OHT treatment during anagen. Error bars represent ± SEM. p<0.05, ** p<0.01, ***p<0.001, ****p< 0.0001. 4-OHT, 4-hydroxytamoxifen; p, postnatal; H&E hematoxylin and eosin.

FIGS. 10A-10F show Epithelial cell-targeted deletion of TSPLR results in increased immune cell infiltration in the skin. FIG. 10A shows experimental timeline (top) and quantification of CD45+ cells in telogen skin (bottom). FIG. 10B shows representative immunostaining images of CD45 (green) in n telogen back skin. FIG. 10C shows experimental timeline (top) and quantification of CD45+ cells in anagen skin (bottom) FIG. 10D shows representative immunostaining of CD45 (green) in anagen back skin. FIG. 10E, FIG. 10F show representative flow cytometry (FIG. 10E) plots and (FIG. 10F) quantification of CD45+ cells in anagen skin. FIG. 10G, FIG. 10H show representative flow cytometry (FIG. 10G) plots and (FIG. 10H) quantification of CD45+CD11b+ cells in anagen skin. Scale bars: 100 µm. Error bars represent ± SEM. p<0.05, ** p<0.01, ***p<0.001, ****p< 0.0001. 4OHT, 4-hydroxytamoxifen; p, postnatal.

FIGS. 11A-11G show Tslpr ablation in HFSC during anagen increased cytotoxic CD8+ T-cell recruitment to HF. FIG. 11A shows exemplary experimental timeline representing 4-OHT treatment during second postnatal hair cycle. FIG. 11B, FIG. 11C show representative flow cytometry plots (FIG. 11B) and quantification (FIG. 11C) of CD45⁺ cells and CD45⁺ CD8⁺ cells in back skin during second telogen. Pregated on live, single cells. FIG. 11D shows representative immunostaining images of CD8 (red) in back skin collected at postnatal day 60. FIG. 11E shows an exemplary experimental timeline depicting 4-OHT treatment during second anagen cycle. FIG. 11F shows quantification of total T cells (left), CD8⁺ T cells (middle) and CD4⁺ T cells (right) in anagen skin by flow cytometry. FIG. 11G shows quantification of CD8+ CXCR3+ cells, (left) and CD8⁺ NKG2D⁺ cells (right) in anagen skin by flow cytometry Scale bars: 100 µm. Error bars represent ± SEM. p<0.05, ** p<0.01, ***p<0.001, ****p< 0.0001. 4OHT, 4-hydroxytamoxifen; p, postnatal.

FIG. 12 shows that subcutaneous TSLP treatment does not result in changes in lymphocyte compartment in skin.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### 1. Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise”, “contain”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “hair disorder” is used in the broadest sense and is inclusive of any disorder wherein one of the signs or symptoms of the disorder includes hair loss.

As used herein the term “skin injury” is used in the broadest sense and is inclusive of any injury to the skin that may be accompanied by hair loss. Exemplary skin injuries include injuries caused by physical stimuli including burns, wounds, punctures, scrapes, scratches, etc.. Additional exemplary skin injuries include injuries due to medical interventions/therapeutic agents, such as skin injury in response to chemotherapy, radiation therapy, and the like.

As used herein, the terms “prevent,” “prevention,” and preventing“ refer to reducing the likelihood of a particular condition or disease state (e.g., hair loss) from occurring in a subject not presently experiencing or afflicted with the condition or disease state. The subject may be at risk of having the condition or disease state (e.g. hair loss).

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

The term “effective amount” or “therapeutically effective amount” refers to an amount sufficient to effect beneficial or desirable biological and/or clinical results.

As used herein, the term “promote” when used in reference to hair growth refers to the ability of an agent (e.g. TSLP) to facilitate any one or more aspects of hair growth. For example, “promoting” hair growth may refer to inducing hair growth, increasing hair growth, preventing cessation of hair growth, etc.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the “subject” is a mammal. In some embodiments, the subject is a human.

Unless otherwise defined, all technical terms used herein have the same meaning as commonly understood by one of ordinary skill in the art to which this disclosure belongs.

### 2. Methods

The present disclosure is based, in part, on the discovery by the inventors on the identification of TSLP as a potent inducer of hair growth in response to skin injury.

In some aspects, provided herein are methods of promoting hair growth in a subject. In some embodiments, provided herein is a method of promoting hair growth in a subject, comprising providing a composition containing a therapeutically effective amount of thymic stromal lymphopoietin (TSLP) to the subject. In some embodiments, the TSLP is recombinant TSLP. In some embodiments, the TSLP is recombinant human TSLP. “Recombinant” refers to a manipulated form of a protein which is encoded by recombinant DNA that has been cloned in a system that supports expression of a gene and translation of messenger RNA, thereby leading to expression of the protein. Recombinant TSLP is commercially available.

The examples herein demonstrate that administration of TSLP promotes hair growth in mice, both following wounding and in the absence of wounding. Methods for determining an appropriate species-specific dose based upon an appropriate dose for another known species are established (see J. Basic Clin. Pharm. March 2016; 7(2): 27-31). In some embodiments, the therapeutically effective amount of TSLP is about 0.01 µg/kg to about 20 µg/kg. In some embodiments, the therapeutically effective amount of TSLP is about 0.025 µg/kg to about 10 µg/kg. In some embodiments, the therapeutically effective amount of TSLP is about 0.5 µg/kg to about 5 µg/kg. In some embodiments, the therapeutically effective amount of TSLP is about 1 µg/kg to about 3 µg/kg. For example, in some embodiments the therapeutically effective amount of TSLP is about 0.01 µg/kg, about 0.02 µg/kg, about 0.03 µg/kg, 0.04 µg/kg, about 0.05 µg/kg, about 0.06 µg/kg, about 0.07 µg/kg, about 0.08 µg/kg, about 0.09 µg/kg, about 0.1 µg/kg, about 0.2 µg/kg, about 0.3 µg/kg, about 0.4 µg/kg, about 0.5 µg/kg, about 0.6 µg/kg, about 0.7 µg/kg, 0.8 µg/kg, about 0.9 µg/kg, about 1.0 µg/kg, about 1.1 µg/kg, about 1.2 µg/kg, about 1.3 µg/kg, 1.4 µg/kg, about 1.5 µg/kg, about 1.6 µg/kg, about 1.7 µg/kg, 1.8 µg/kg, about 1.9 µg/kg, about 2.0 µg/kg, about 2.1 µg/kg, about 2.2 µg/kg, about 2.3 µg/kg, 2.4 µg/kg, about 2.5 µg/kg, about 2.6 µg/kg, about 2.7 µg/kg, about 2.8 µg/kg, about 2.9 µg/kg, about 3.0 µg/kg, about 3.1 µg/kg, about 3.2 µg/kg, about 3.3 µg/kg, about 3.4 µg/kg, about 3.5 µg/kg, about 3.6 µg/kg, about 3.7 µg/kg, about 3.8 µg/kg, about 3.9 µg/kg, about 4.0 µg/kg, about 4.1 µg/kg, about 4.2 µg/kg, about 4.3 µg/kg, about 4.4 µg/kg, about 4.5 µg/kg, about 4.6 µg/kg, about 4.7 µg/kg, about 4.8 µg/kg, about 4.9 µg/kg, or about 5.0 µg/kg. As another example, in some embodiments the therapeutically effective amount of TSLP is about 5.0 µg/kg, about 5.5 µg/kg, about 6 µg/kg, about 6.5 µg/kg, about 7 µg/kg, about 7.5 µg/kg, about 8.0 µg/kg, about 8.5 µg/kg, about 9 µg/kg, about 9.5 µg/kg, about 10 µg/kg, about 10.5 µg/kg, about 11 µg/kg, about 11.5 µg/kg, about 12 µg/kg, about 12.5 µg/kg, about 13 µg/kg, about 13.5 µg/kg, about 14 µg/kg, about 14.5 µg/kg, or about 15 µg/kg. As another example, in some embodiments the therapeutically effective amount of TSLP is about 15 µg/kg, about 15.5 µg/kg, about 16 µg/kg, about 16.5 µg/kg, about 17 µg/kg, about 17.5 µg/kg, about 18 µg/kg, about 18.5 µg/kg, about 19 µg/kg, about 19.5 µg/kg, or about 20 µg/kg.

In some embodiments, the composition is provided to the subject parenterally (e.g. by injection, including intramuscular, subcutaneous, transdermal, intravenous, etc.). In some embodiments, the composition is provided to the subject topically. In some embodiments, the composition is provided to the subject subcutaneously. In some embodiments, the composition is provided to the subject at the site of hair loss.

In some embodiments, the subject has experienced a skin injury. For example, in some embodiments the subject as experienced a skin injury such as a burn or a wound that has caused hair loss in the subject, and the composition is provided to the subject to promote hair growth at the site of hair loss. For example, in some embodiments the subject has experienced a skin injury and the composition is provided to the subject subcutaneously at the site of skin injury, thereby promoting hair growth at the site of the skin injury.

In some embodiments, the subject has a hair disorder. Exemplary hair disorders include alopecia (e.g. androgenetic alopecia, alopecia areata, cicatricial alopecia, lichen planopilaris, frontal fibrosing alopecia, central centrifugal cicatricial alopecia, traction alopecia), male pattern hair loss, female pattern hair loss, telogen effluvium, anagen effluvium, tinea capitis, discoid lupus erythematosus, folliculus decalvans, dissecting cellulitis of the scalp, loose anagen syndrome, trichotillomania, hypotrichosis. In some embodiments, the hair disorder is alopecia. For example, in some embodiments the hair disorder is alopecia areata. As another example, in some embodiments the hair disorder is androgenetic alopecia.

In some embodiments, the subject has alopecia and the composition is provided to the subject (e.g. subcutaneously) to promote hair growth in the subject. In some embodiments, the subject has alopecia and the subject has experienced a skin injury (e.g. microneedling). For example, a subject with alopecia may be undergoing microneedling as part of a treatment plan for alopecia, and the treatment plan may further comprise providing to the subject a composition comprising a therapeutically effective amount of TSLP in conjunction with the microneedling. The composition may be applied before, during, and/or after microneedling to promote hair growth. In some embodiments, the composition is applied directly to a wound site (e.g. a microneedle site).

In some embodiments, the subject is a mammal. In some embodiments, the subject is a human. The human subject may be a pediatric subject (e.g. less than 18 years of age) or an adult subject (e.g. 18 years or older).

In some embodiments, the composition is provided to the subject a single time. For example, the composition may be provided to the subject a single time to promote hair growth. In some embodiments, the composition is provided to the subject multiple times. The timing and intervals between consecutive doses of the composition can vary depending on the subject, the degree of hair loss, the cause of hair loss, and the like. In some embodiments, the composition is provided to the subject once daily, once every other day, once every three days, once every four days, once every five days, once every six days, once every week (i.e. weekly), once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 3 weeks, monthly, yearly, etc.

In some aspects, provided herein are methods of preventing chemotherapy-induced hair loss in a subject. Hair loss during chemotherapy occurs in 99% of breast cancer patients. Limited treatments are available, with 84% of patients using a wig during the first year of chemotherapy. General chemotherapy induces alopecia in ~65% of patients. Chemotherapy targets the rapidly dividing hair follicles. TSLP is shown herein to play a role in promoting hair growth, and suppression of TSLP is shown herein to arrest/delay hair growth. By temporarily preventing hair growth and thereby reducing the rapidly dividing activity at the follicle, chemotherapy may not cause hair loss as it would no longer target hair during treatment. Accordingly, in some embodiments provided herein is a method of preventing chemotherapy-induced hair loss in a subject comprising providing a composition containing a therapeutically effective amount of an inhibitor of thymic stromal lymphopoietin (TSLP) to the subject. In some embodiments, the composition comprising the inhibitor of TSLP is provided to the subject before, during, and/or after chemotherapy, thereby preventing chemotherapy-induced hair loss in the subject.

In some embodiments, the inhibitor of TSLP comprises an antibody or a fragment thereof or a small molecule inhibitor. In some embodiments, the antibody comprises Tezepelumab (AMG-157/MEDI9929). In some embodiments, the composition comprising the inhibitor of TSLP (e.g. anti-TSLP antibody) is provided to the subject before chemotherapy. In some embodiments, the composition is provided to the subject concurrently with chemotherapy. In some embodiments, the composition is provided to the subject after therapy. In some embodiments, the composition is provided to the subject about 2 weeks, about 13 days, about 12 days, about 11 days, about 10 days, about 9 days, about 8 days, about 1 week, about 6 days, about 5 days, about 4 days, about 3 days, about 2 days, or about 1 day prior to chemotherapy. In some embodiments, the composition is provided to the subject before chemotherapy (e.g. less than about 2 weeks before chemotherapy) and concurrently with chemotherapy.

Any suitable dose of the inhibitor of TSLP may be provided to the subject. In some embodiments, the inhibitor comprises Tezepelumab, and the dose is about 1 µg/kg to about 100 mg/kg. For example, in some embodiments the inhibitor comprises Tezepelumab, and the dose is about 1 µg/kg to about 100 mg/kg, about 5 µg/kg to about 75 mg/kg, about 10 µg/kg to about 50 mg/kg, about 25 µg/kg to about 45 mg/kg, about 50 µg/kg about 40 mg/kg, about 75 µg/kg to about 45 mg/kg, about 100 µg/kg to about 40 mg/kg, about 150 µg/kg to about 35 mg/kg, about 200 µg/kg to about 30 mg/kg, about 300 µg/kg to about 25 mg/kg, about 400 µg/kg to about 20 mg/kg, about 500 µg/kg to about 15 mg/kg about 750 µg/kg to about 10 mg/kg, or about 1 mg/kg to about 5 mg/kg. In some embodiments, the composition is provided to the subject parenterally (e.g. by injection, including intramuscular, subcutaneous, transdermal, intravenous, etc.). In some embodiments, the composition is provided to the subject topically. In some embodiments, the composition is provided to the subject subcutaneously. In some embodiments, the composition is provided to the subject at the site or predicted site of hair loss.

### EXAMPLES

### Example 1

Skin tissue regeneration after injury involves production and integration of signals by stem cells residing in hair follicles (HFSCs). Much remains unknown about how specific wound-derived factors modulate stem cell contribution to hair growth. The data provided herein demonstrate that thymic stromal lymphopoietin (TSLP) is produced in response to skin injury and during the anagen phase of the hair cycle. Intradermal injection of TSLP promoted wound-induced hair growth (WIHG), whereas neutralizing TSLP receptor (TSLPR) inhibited WIHG. Using flow cytometry and fluorescent immunostaining, it was found that TSLP promoted proliferation of transit amplifying cells. Lgr5CreER-mediated deletion of Tslpr in HFSCs inhibited both wound-induced and exogenous TSLP-induced hair growth. The data highlight a novel function for TSLP in regulation of hair follicle activity during homeostasis and wound healing.

### Results

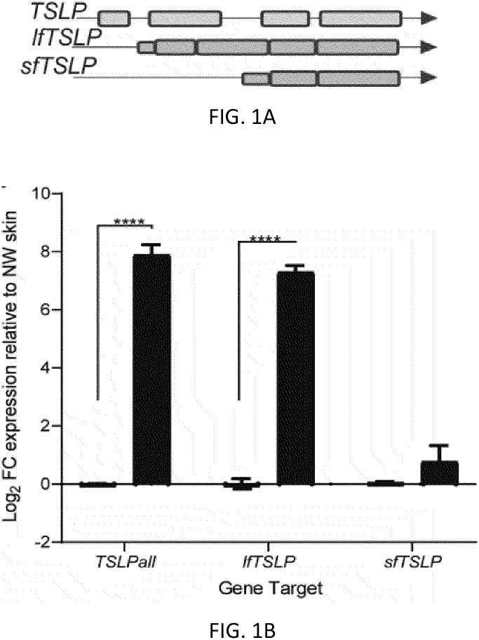

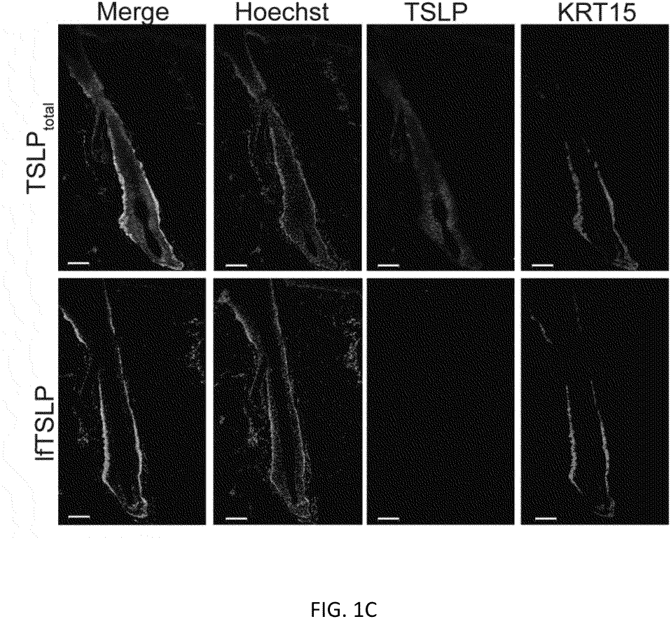

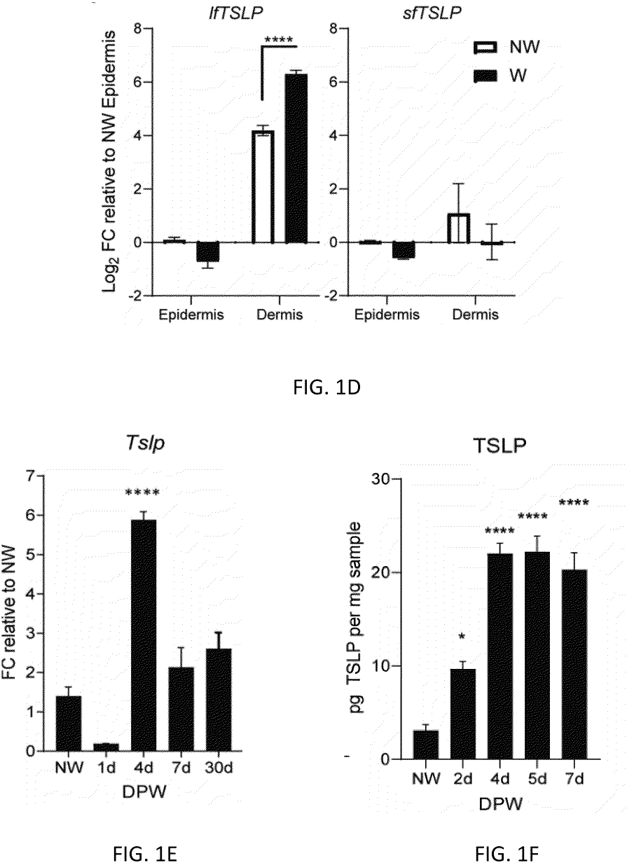

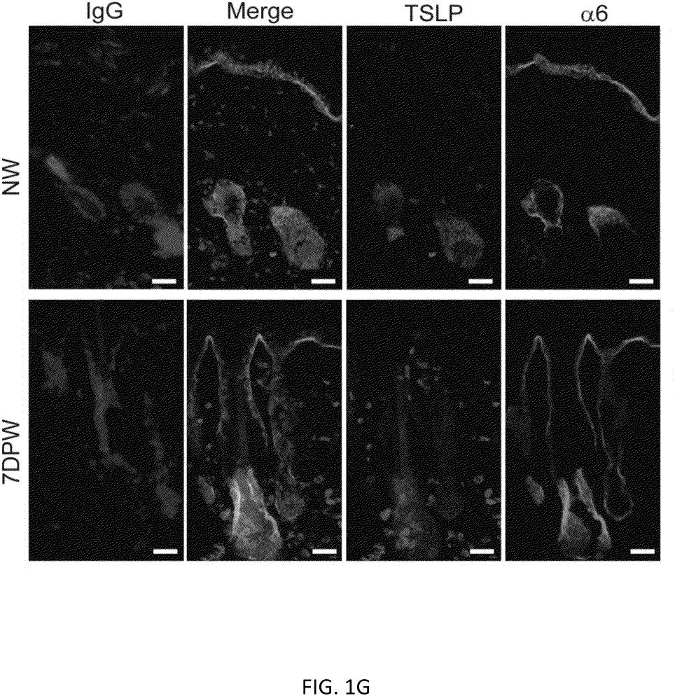

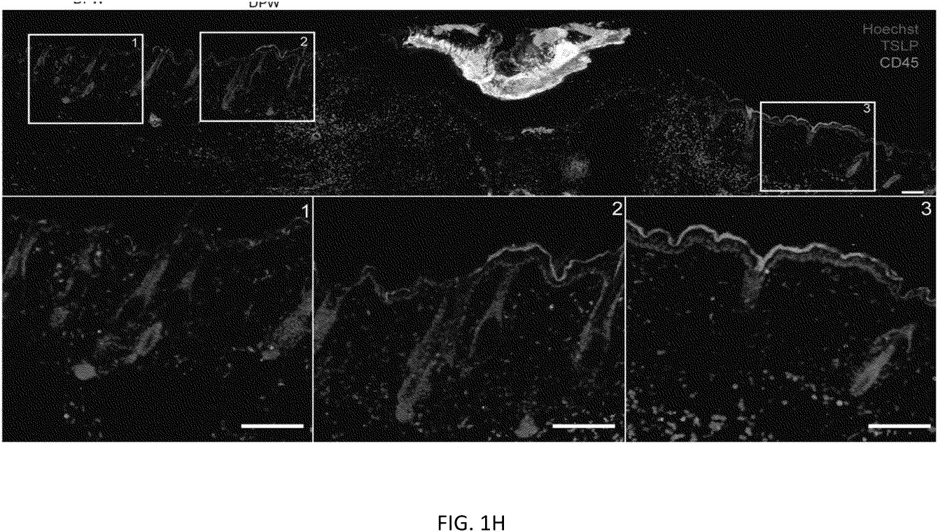

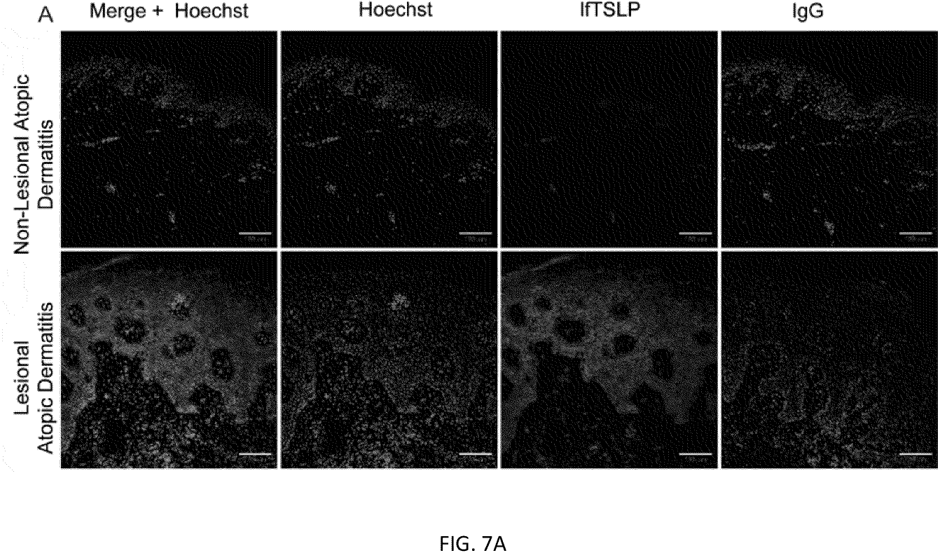

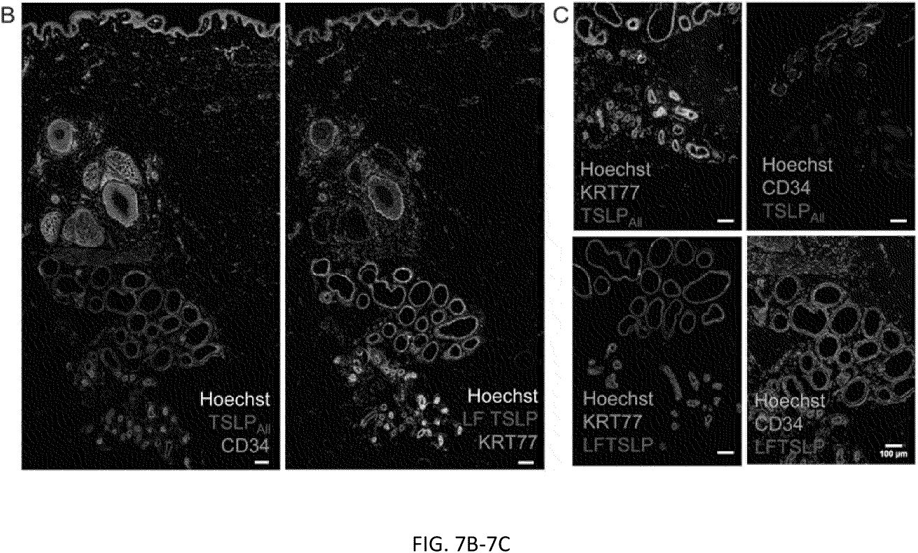

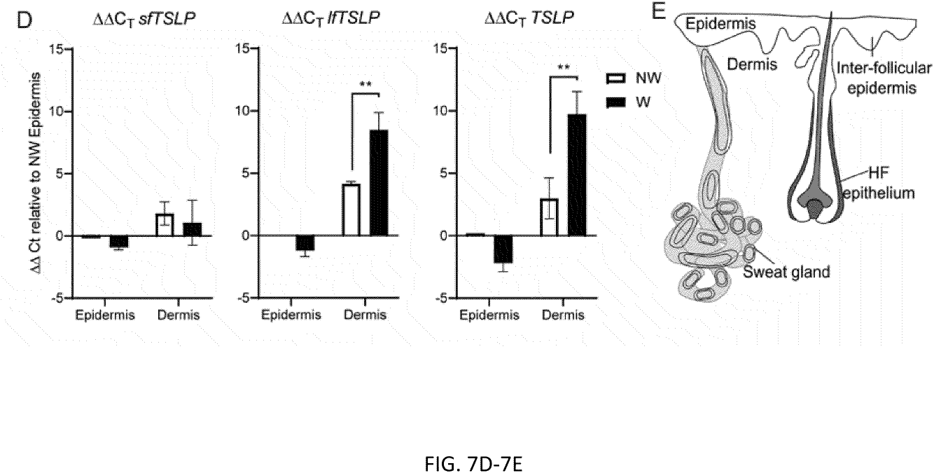

TSLP is produced in the skin in response to injury: There are two variants of human TSLP whose expressions are dictated by two putative promoter regions with different open reading frames that share a C-terminal region (FIG. 1A). Long form TSLP (lfTSLP) is linked to type-2 immune responses and highly induced in pathological conditions such as allergic diseases. Short form TSLP (sfTSLP) is absent in mice but constitutively expressed in human epidermis. Using quantitative reverse transcription polymerase chain reaction (qPCR) with primers designed to discriminate between the two variants, it was found that only lfTSLP was consistently upregulated in human skin 24 hours post scratch-wounding ex vivo (FIG. 1B). To distinguish lfTSLP from sfTSLP at the protein level, IfTSLP-specific polyclonal antibodies targeting the N-terminal region unique to lfTSLP were generated. The specificity of the lfTSLP antibody was validated and confirmed up-regulation of lfTSLP in atopic dermatitis lesions using immunofluorescence (FIG. 7A). lfTSLP was undetectable in healthy human skin, but TSLPtotal, as detected by the antibody that recognizes both variants, was abundantly expressed in the hair follicle (FIG. 1C) and epidermis (FIGS. 7B-C), indicating that sfTSLP, but not lfTSLP, is expressed in the normal skin. qPCR analysis of enzymatically separated epidermal and dermal parts of wounded skin revealed that lfTSLP was most significantly upregulated in the dermal part that contained dermis and hair follicles of both human and mouse skin (FIGS. 1D, 7D-E). Interestingly, TSLP expression was largely restricted to hair follicles, sebaceous glands, and sweat glands in normal human skin as shown by immunostaining (FIGS. 7B-E). Analysis of TSLP expression across different murine tissues revealed that TSLP expression was highest in the skin compared to other tissues including lymphoid organs.

TSLP functions via its heterodimeric receptor comprised of TSLPR (encoded by Crlf2) and IL7Ra; the receptor complex is highly conserved between human and mouse. Through analysis of existing transcriptomic data sets of full-thickness healing mouse wounds, it was noted that Tslp, Il7ra, and Crlf2 were all increased after wounding. Consistently, qPCR-based time-course analysis revealed that Tslp mRNA peaked around 4 days after wounding (FIG. 1E). Analysis by ELISA verified that TSLP protein level remained elevated for at least 7 days after wounding (FIG. 1F). TSLP was detected in telogen and early anagen hair follicles of non-wounded skin and in hair follicles of skin 7 days post-wounding (FIGS. 1G-H). TSLP is highly expressed the dermal cells that likely included fibroblasts and immune cells, as indicated by positive immunostaining of TSLP in both CD45+ and CD45- cell populations. In addition, it was found that TSLP is readily detected in hair follicle bulge and hair germs (FIG. 1G). The notable expression pattern of TSLP in HF keratinocytes and surrounding stromal cells coinciding with anagen entry suggests that TSLP plays a role in anagen induction during tissue regeneration.

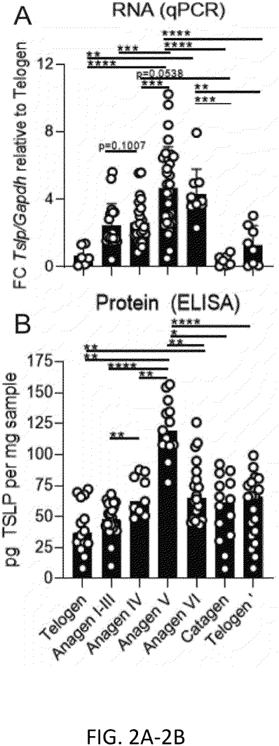

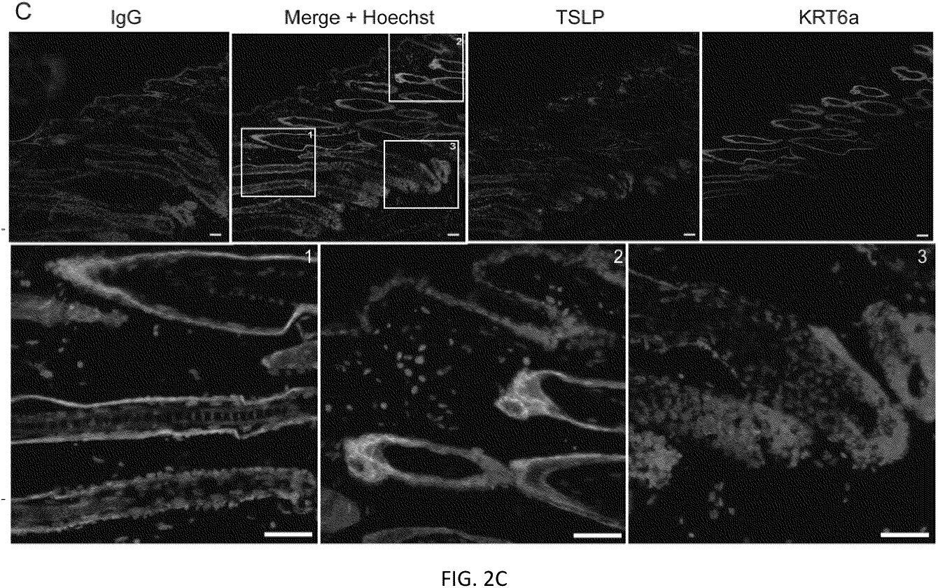

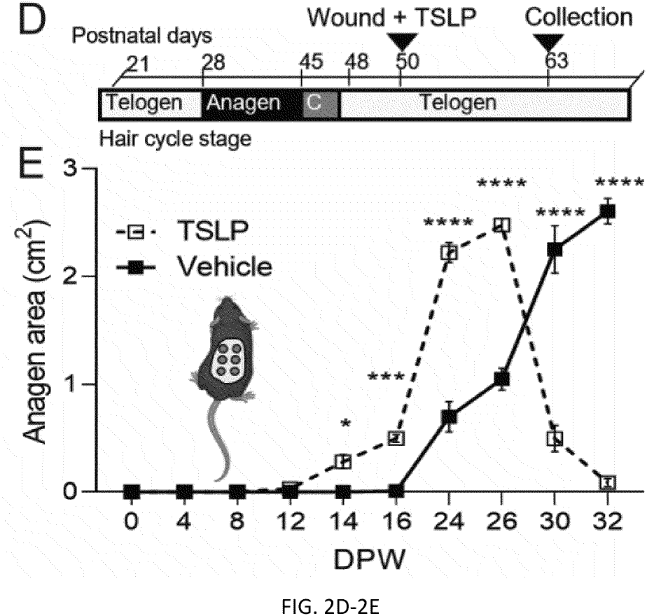

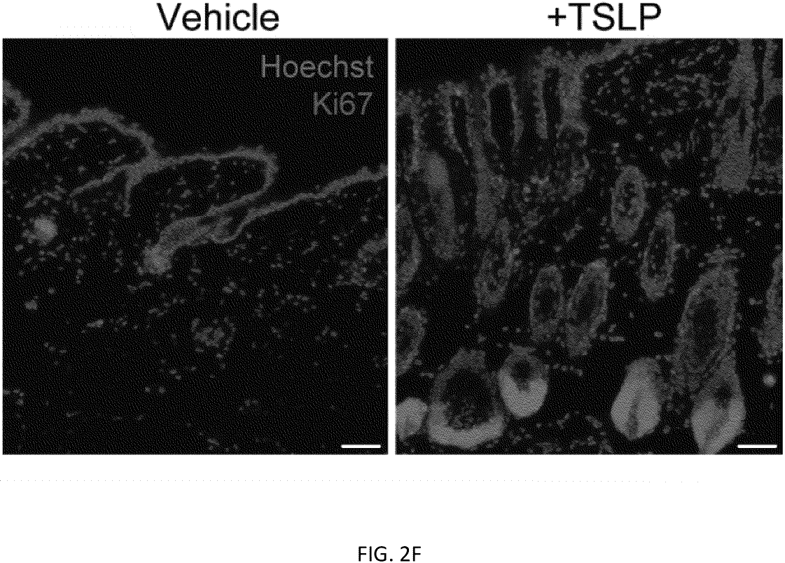

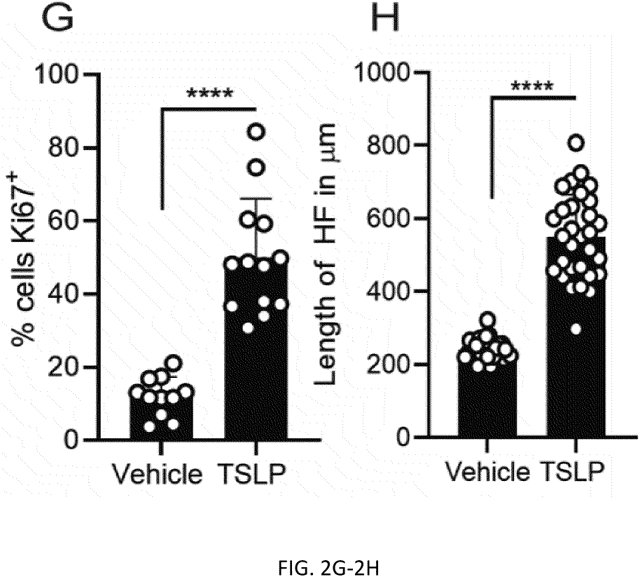

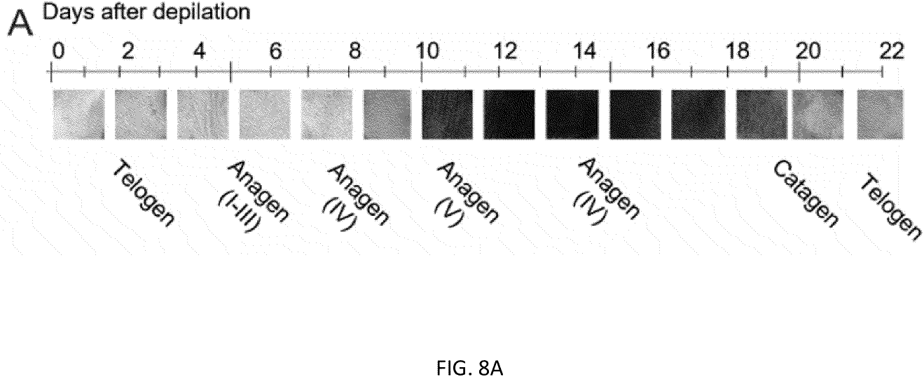

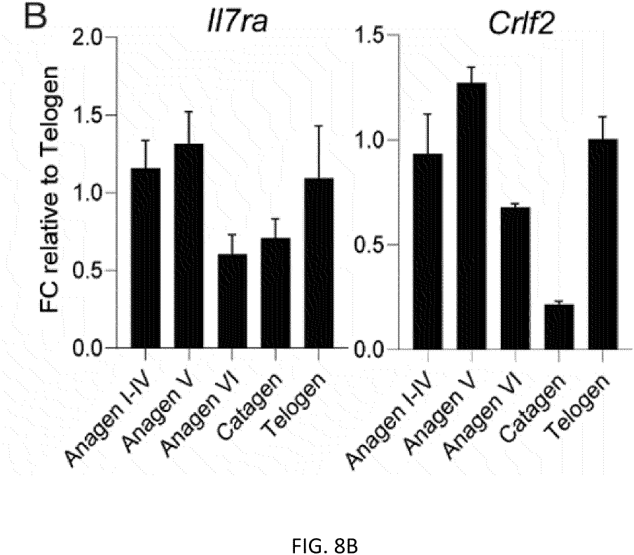

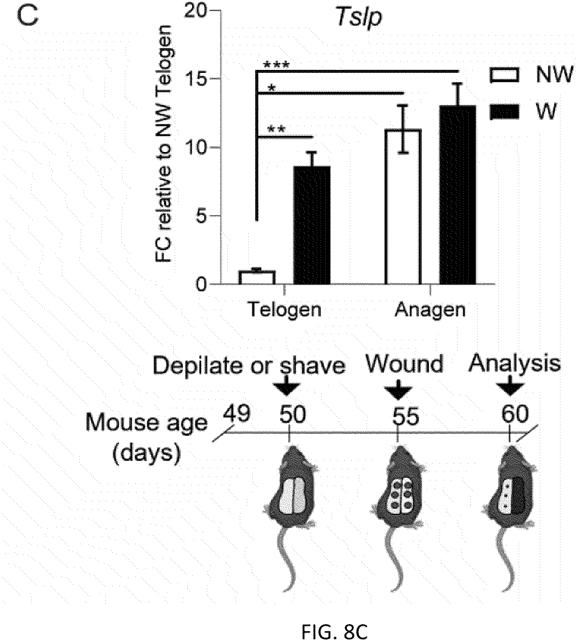

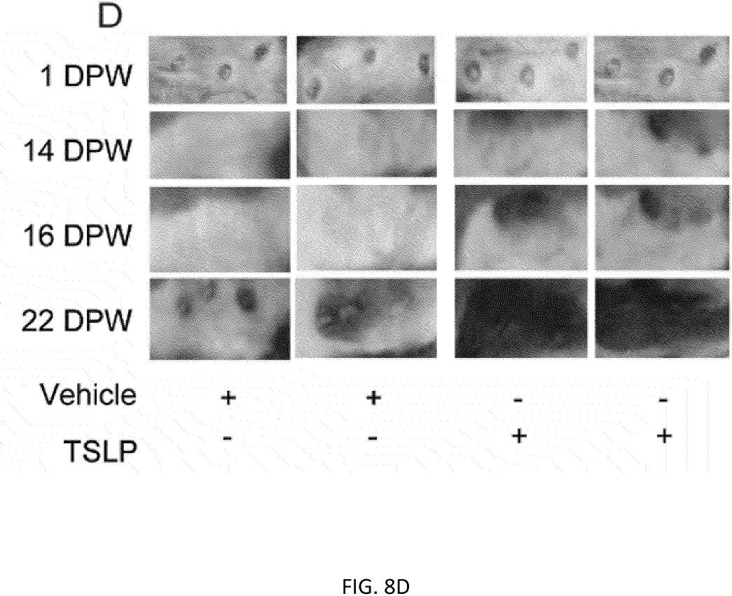

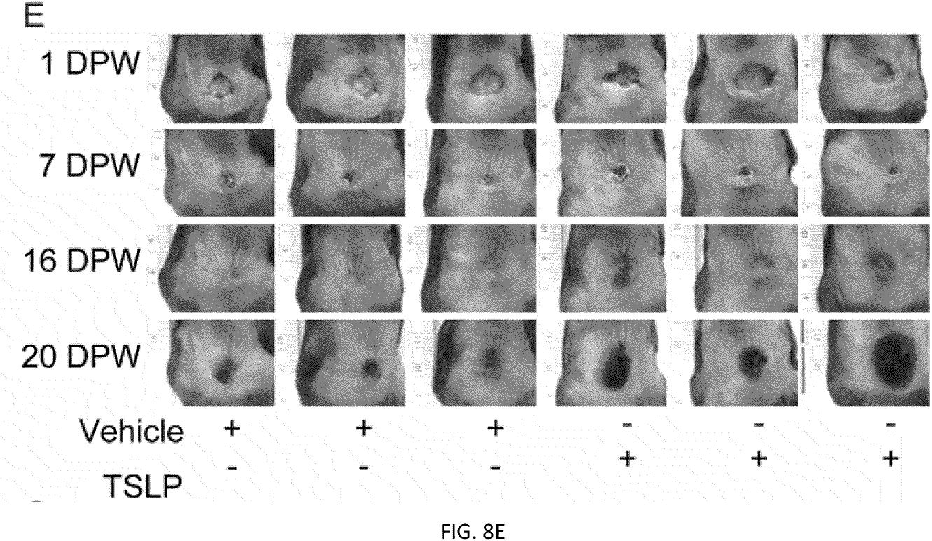

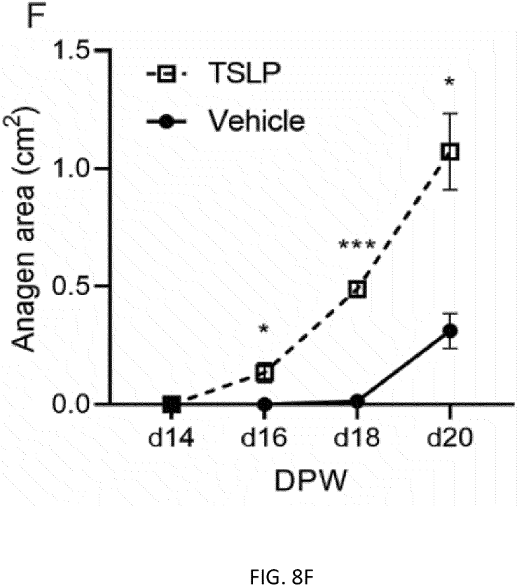

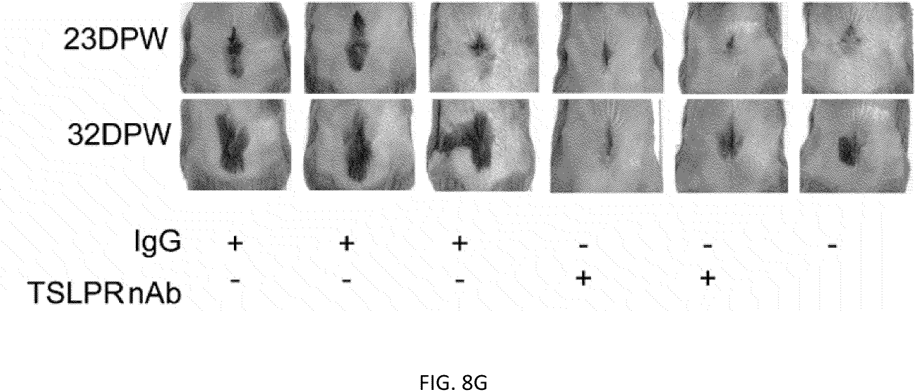

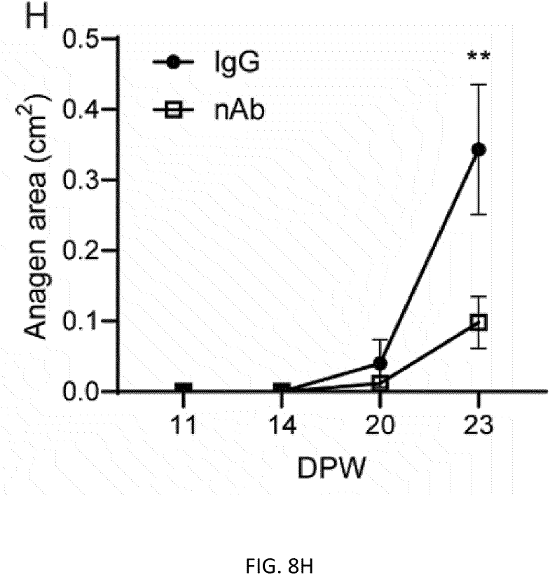

TSLP is expressed throughout the hair cycle and accelerates onset of wound-induced hair growth: Stem cells in hair follicles mobilize after injury and aid regeneration of hair follicles, sebaceous glands, and the epidermis. In particular, skin injury triggers activation of stem cells in surrounding telogen hair follicles to enter into hair cycling, a phenomenon understood as wound-induced hair growth (WIHG) that begins 7 days post wounding. To evaluate whether TSLP plays a role in hair cycle activation, TSLP expression was first profiled throughout the hair cycle following depilation-induced entry of anagen phase. Hair cycling was monitored based on skin pigmentation as a defined temporal criterion (FIG. 8A). qPCR and ELISA revealed that TSLP was upregulated throughout the hair cycle, reaching peak expression during mid-anagen when hair follicles extend deeper into the tissue, and then downregulated to lowest expression during catagen (FIGS. 2A-B). TSLP immunostaining was most prominent in cells of the outer root sheath and in the hair follicle bulge during mid-anagen (FIG. 2C). In contrast, Crlf2 and Il7ra expressions did not exhibit significant changes throughout the hair cycle (FIG. 2A, FIG. 8B). To further determine Tslp transcriptional changes in wound-induced hair cycles, mouse skin was wounded in synchronized telogen and anagen phases and collected tissues 5 days after wounding. Tslp expression increased in response to injury and peaked in anagen skin; however, wounding anagen skin did not exhibit added effects to the already elevated Tslp expression (FIG. 8C). These data suggest that TSLP functions locally in the hair follicle microenvironments to promote tissue regeneration.

To evaluate whether TSLP has a functional role in WIHG onset and accelerates hair follicle proliferation, recombinant TSLP was administered directly to the wound bed of small (4-mm diameter) and large (12-mm diameter) excisional punch wounds at the time of wounding. It was found that wounds treated with TSLP consistently showed accelerated WIHG, as measured by the area of skin that entered anagen (FIGS. 2D-E, 8D-F). Next, TSLPR signaling was interrupted using neutralizing antibodies (nAb) administered directly to the wound bed immediately after wounding and again 4 days after wounding. TSLPR nAb treated mice showed reduced hair growth when compared to the IgG control (FIGS. 8G-H). Together, these data indicate that exogenous TSLP is sufficient and required for driving WIHG in large and small full-thickness wounds.

Tissue sections were next examined from 13-day old wound beds treated with 100 ng TSLP or 0.01% BSA in PBS to determine how TSLP altered behavior of hair follicle and other skin cells. TSLP-treated tissue showed morphological changes indicative of anagen entry 13 days after wounding and increased expression of the cell proliferation marker Ki67, compared to that of the control group (FIGS. 2F-H). Similarly, TSLP treatment resulted in increased hair follicle length, epidermal thickness, and diameter of the hair follicle bulge (FIGS. 2H, 8I). Thus, TSLP delivery to wounds increased cellular proliferation and accelerated hair cycle entry into anagen and growth.

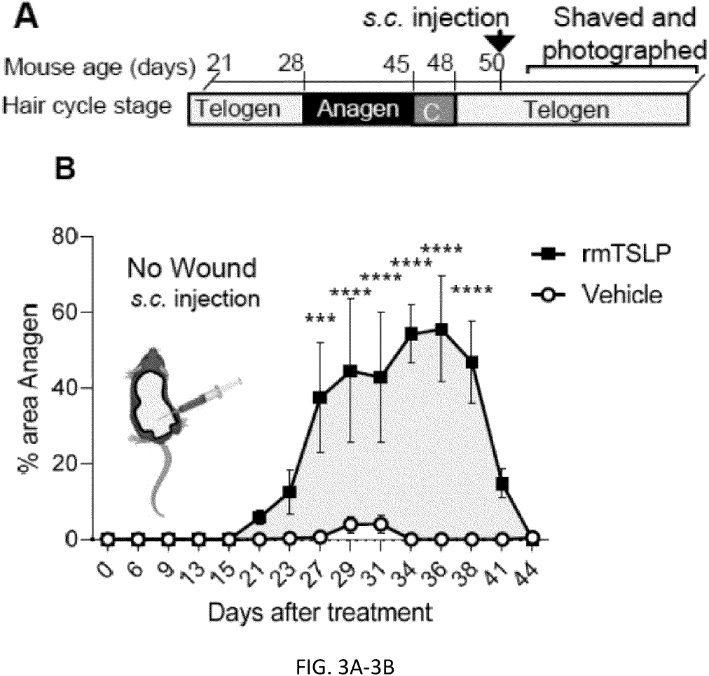

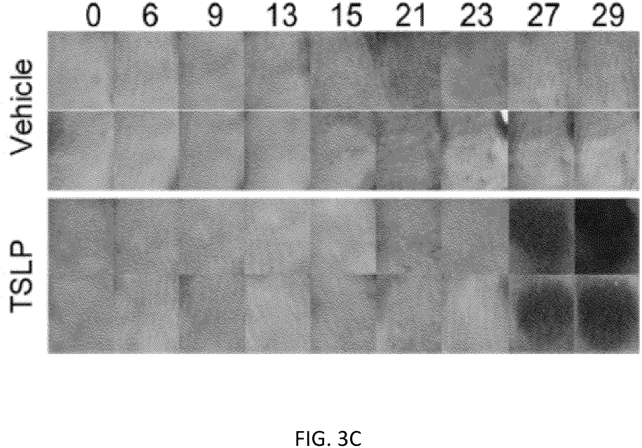

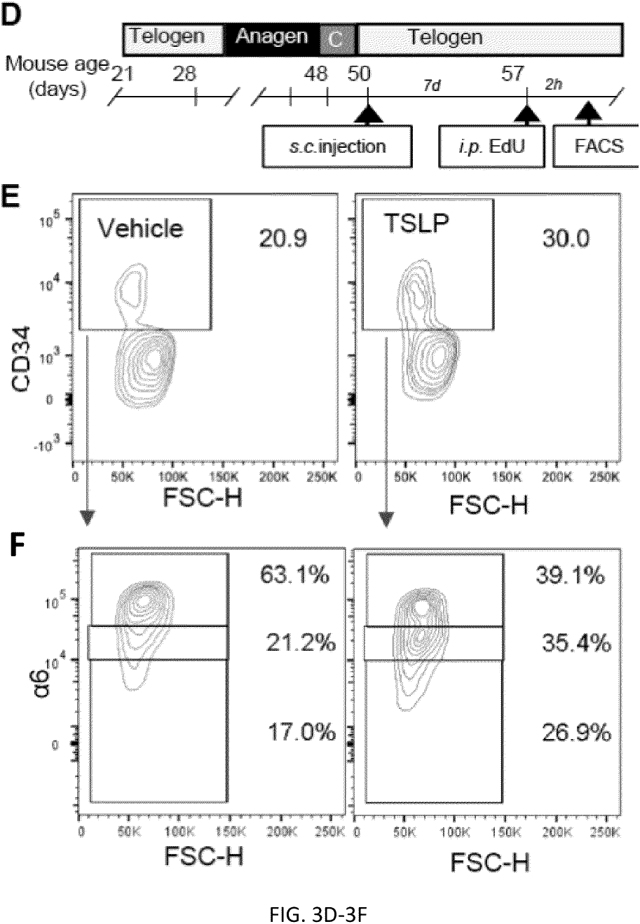

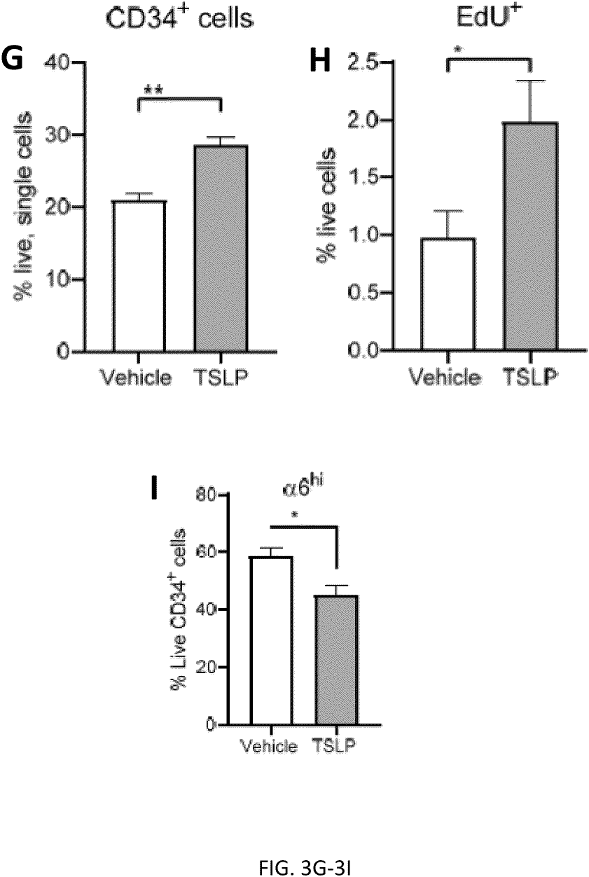

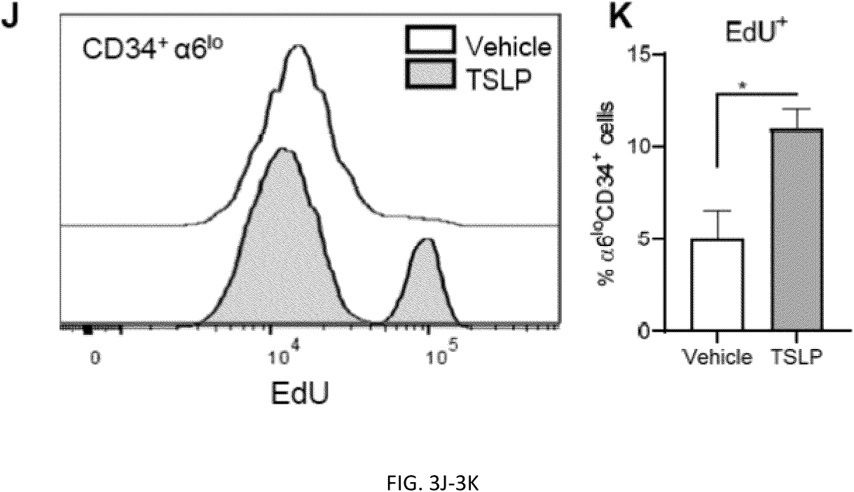

TSLP expands CD34+ITGα6lo transit amplifying cell (TAC) population: Next, it was investigated whether TSLP is sufficient to drive hair cycling in the absence of injury. Mice were given subcutaneous (s.c.) injections of recombinant TSLP or vehicle control at second telogen (FIG. 3A). TSLP treatment alone was sufficient to drive hair cycling in wild-type mice (FIGS. 3B-C); interestingly, s.c. TSLP treatment resulted in hair growth temporally consistent with normal wound-induced hair growth. These findings indicate that hair follicle stem cells may be directly responsive to local TSLP. To investigate this, mice were treated with TSLP, and 7 days later, pulsed animals with 5-ethynyl-2′-eoxyuridine (EdU) for 2 hours to label proliferating cells. Tissues were then collected and analyzed the frequency of cells expressing stem cell markers of the hair germ (LGR5), hair bulge (CD34), and EdU incorporation (FIG. 3D). FACS analysis confirmed that TSLP-treatment resulted in the expansion of LGR5+ HFSC, CD34+ progenitors, and total EdU+ cells (FIGS. 3E, 3G, and 3H, S4A-E). Interestingly, EdU+ cells were enriched in the CD34+ITGα6lo population (FIG. 3F, FIGS. 3H-K), previously characterized as TAC cells.

Two distinct CD34+ stem cell populations exist within the hair follicle distinguished by expression intensity of integrin α6 (ITGα6); lower levels of ITGα6 indicate suprabasal positioning of cells derived from basal progenitors and higher levels of ITGα6 mark cells attached to the basement membrane. CD34+ITGα6lo cells are early progeny of ITGα6hi basal bulge stem cells. Both CD34+ITGα6hi and CD34+ITGα6lo cells manifest self-renewal properties of stem cells: they withstand multiple passages in tissue culture ex vivo and can give rise to interfollicular epidermis and hair. By stratifying CD34+ cells by ITGα6 expression, it was observed that TSLP treated skin contained a unique CD34+ITGα6lo cell population that was not present in the vehicle-treated skin (FIG. 3F, FIG. 3I). Further analysis of this ITGα6lo cell population revealed increased EdU incorporation, indicating that the CD34+ ITGα6lo cells are highly proliferative compared to CD34+ ITGα6hi and CD34- cells (FIGS. 3J-K). Together, these results indicate that TSLP drives the amplification of TACs, highlighting TSLP as a core growth factor of HF niche.

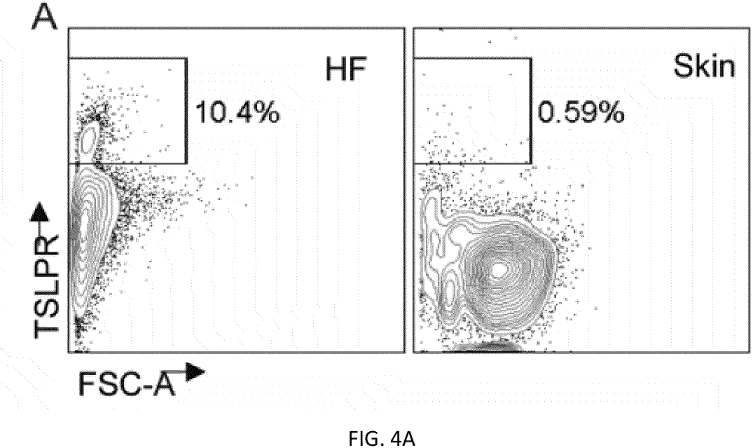

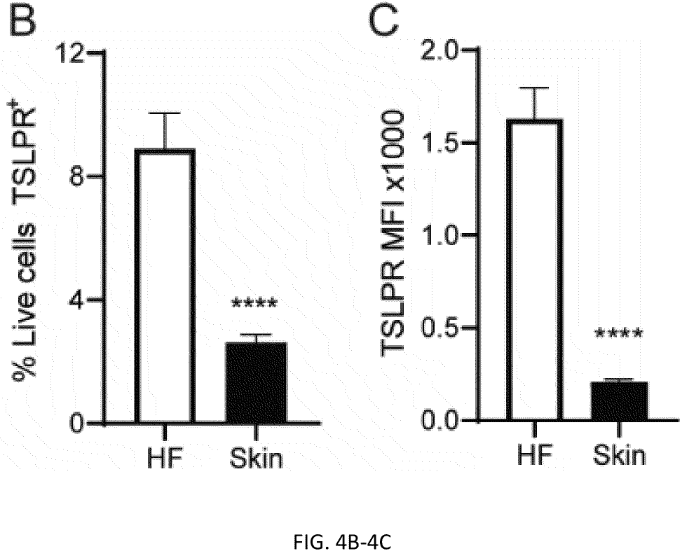

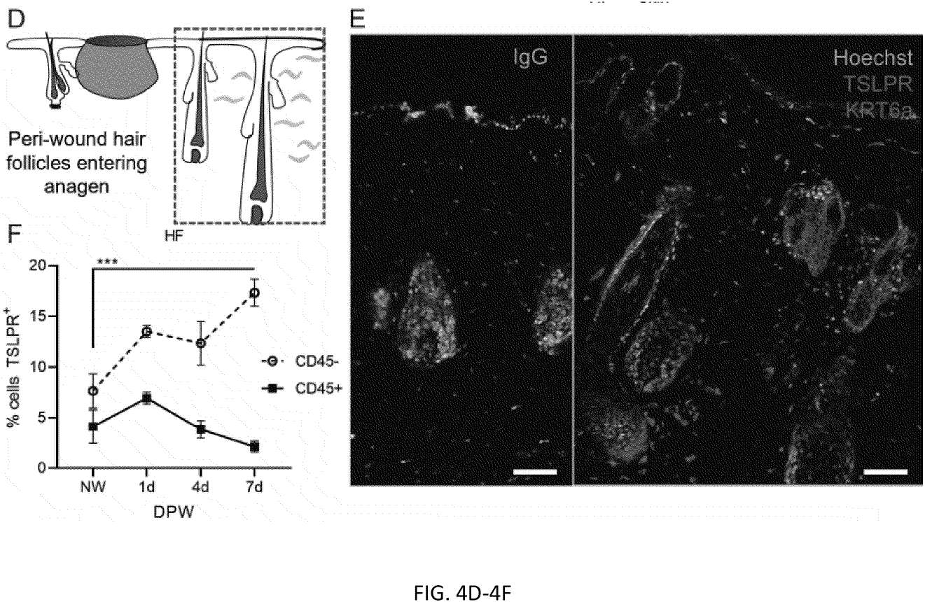

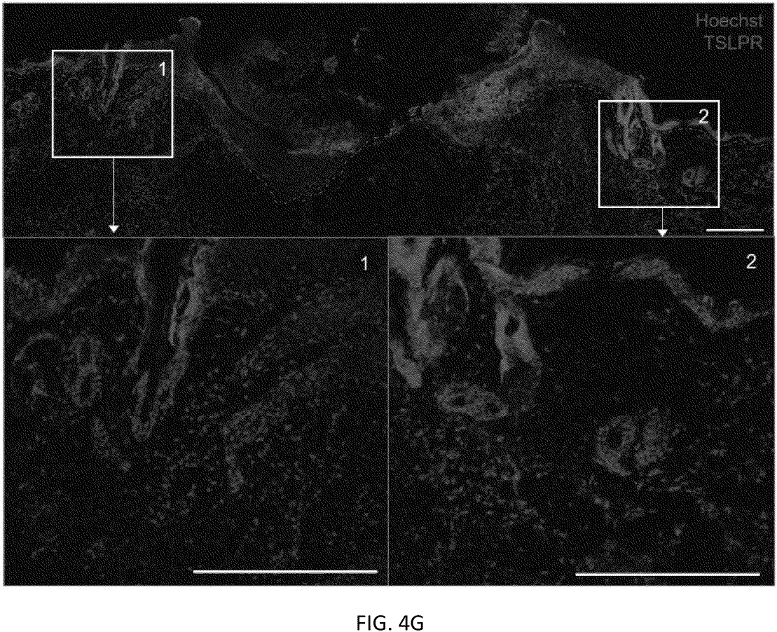

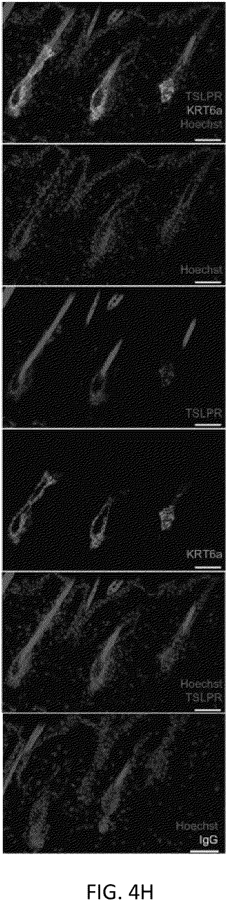

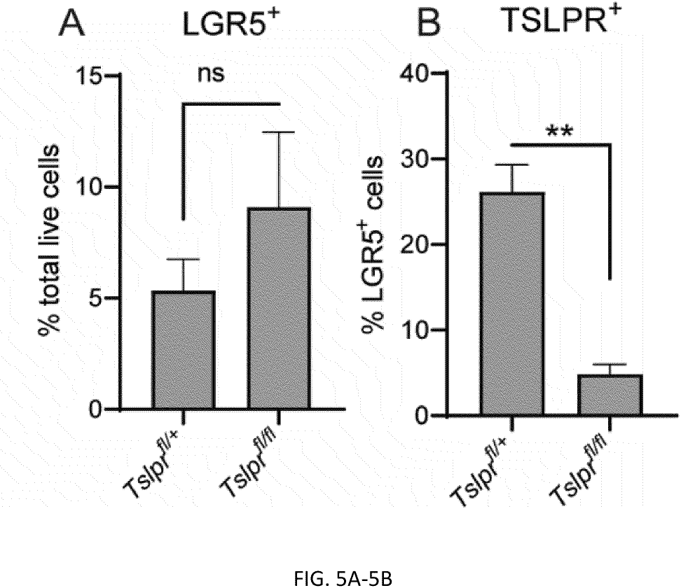

Murine epithelial cells express TSLPR. It was next sought to identify cell types that mediate accelerated hair growth in response to TSLP. Flow cytometry analysis of tissues revealed that hair follicles were enriched with TSLPR+ cells compared to whole skin (FIGS. 4A-C). Immunostaining of 7-day skin wound beds revealed that cells in wound-adjacent hair follicles expressed TSLPR (FIGS. 4D-E). Since TSLP is a cytokine with immunomodulatory function, it was investigated whether TSLPR expression changes occur in immune cells and keratinocytes during wound healing. Flow cytometry was then used to quantify TSLPR expression in CD45+ immune and CD45- non-immune cell compartments. Unexpectedly, it was found that TSLPR expression was greatest on CD45- cells in hair follicles of non-wounded tissue and CD45-TSLPR+ cell population expanded during wound healing (FIG. 4F). Immunostaining was then performed to determine the spatial orientation of TSLPR+ cells around the wound bed and in non-wounded telogen skin. TSLPR expression was largely localized to the outer root sheath of the hair follicle in both non-wounded and wounded skin, though wounded skin also exhibited strong TSLPR staining in the leading edge of the epidermis in comparison to non-wounded epidermis (FIGS. 4G-H). Notably, LGR5+ progenitors in the hair follicle contribute to the leading wound edge during tissue regeneration. The strong TSLPR signal on epithelial cells in and around the hair follicle bulge area suggests a role for TSLPR in hair follicle responses to injury.

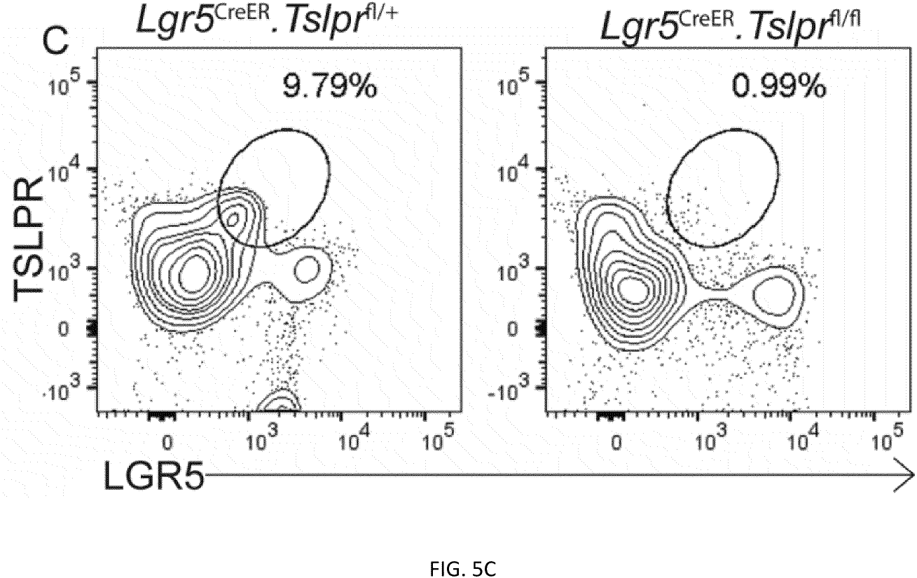

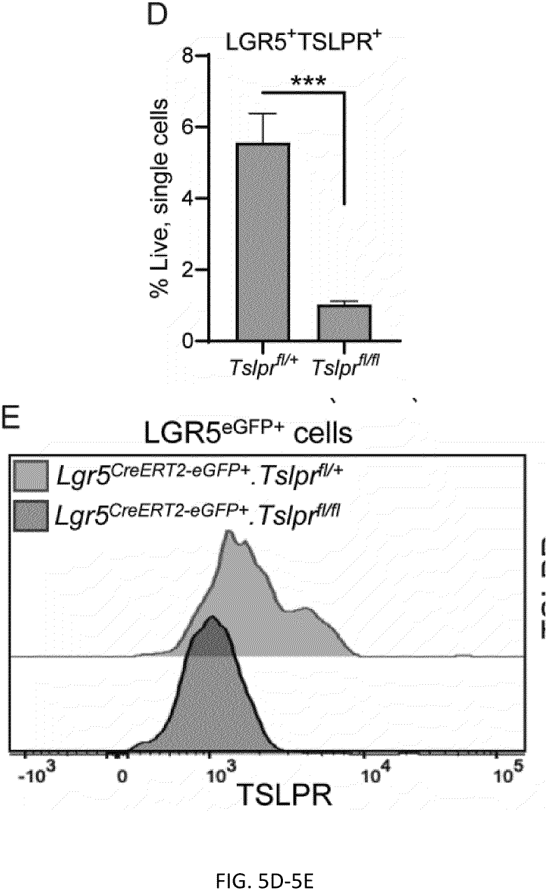

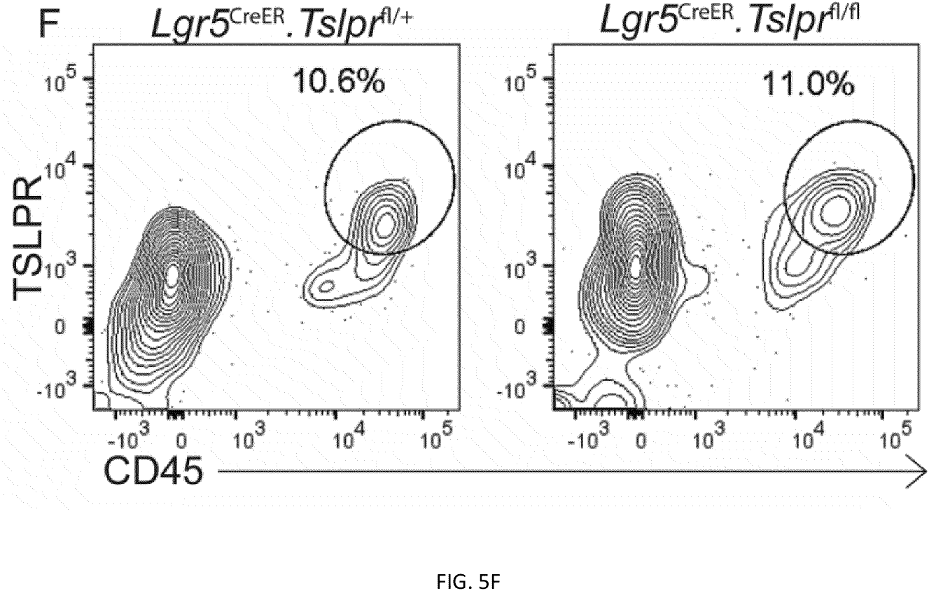

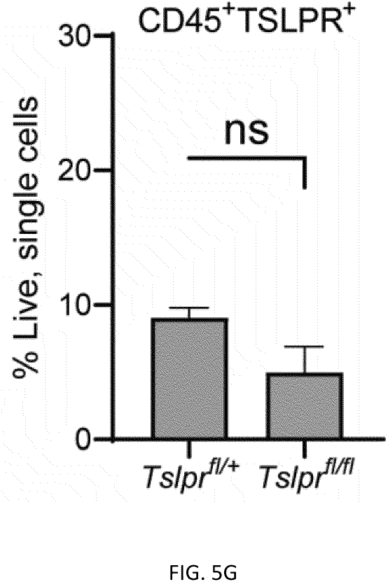

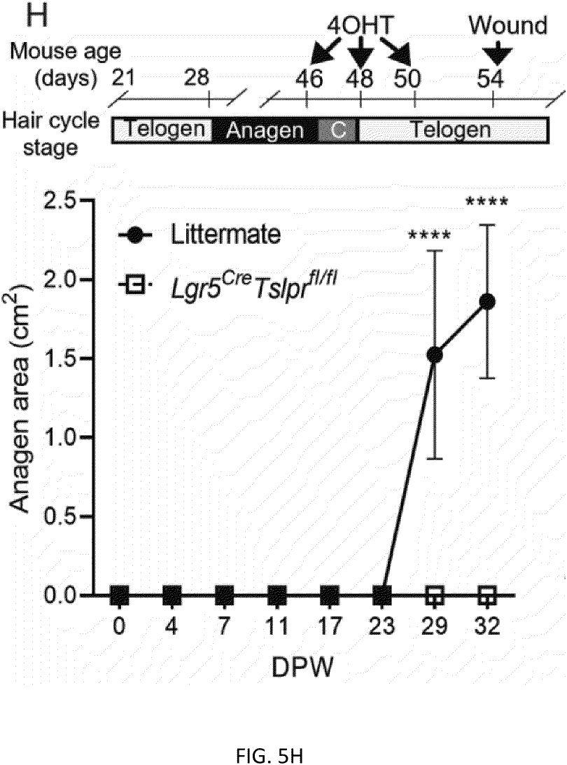

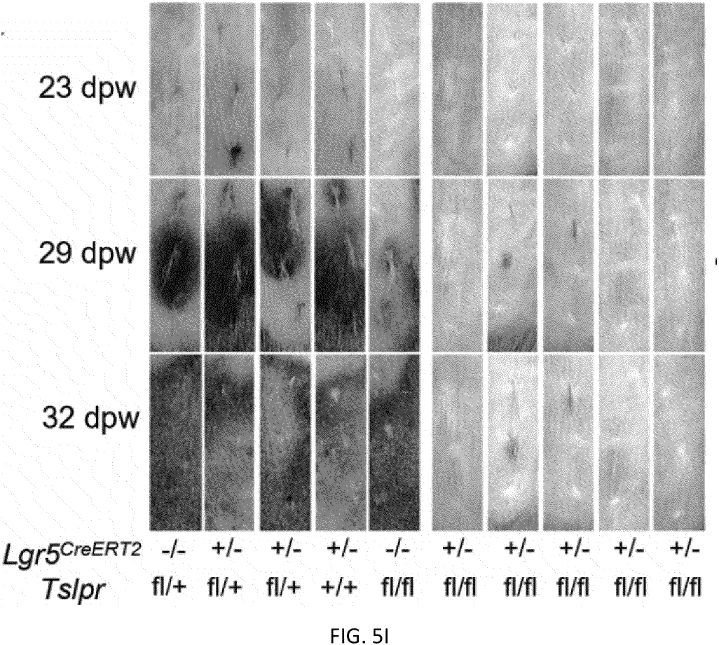

TSLPR expression in LGR5+ HFSC during WHIG. To investigate whether TSLP acts directly on HFSC to expand the TAC compartment, Lgr5CreER mice were crossed with Tslprfl/fl mice. Epithelial cell-targeted ablation of Tslpr was achieved by 4 consecutive daily topical treatments of 4-hydroxytamoxifen (4OHT) and confirmed by flow cytometry which showed significant knockdown of TSLPR expression in LGR5+ cells (FIGS. 5A-E), but not in CD45+ hematopoietic cells (FIGS. 5F-G). A difference in the frequency of LGR5+ cells was not detected in 4OHT-induced mutant mouse skin compared to littermate controls, indicating that TSLPR loss did not deplete LGR5+ stem cell population. To address whether LGR5+ cells require TSLPR for WIHG, Lgr5CreER.Tslprfl/fl mice were treated and their littermate controls with topical 4-OHT to induce Tslpr ablation prior to wounding at second telogen (FIG. 5H, top). Control mice showed hair growth by day 23; in sharp contrast, Lgr5CreER.Tslprfl/fl mice did not exhibit signs of WIHG for at least 32 days after wounding (FIGS. 5H-I). It was next asked whether TSLPR accelerates WIHG in response to exogenous TSLP (FIG. 5J). Animals were treated with three tropical doses of 4OHT and then wounded telogen skin along with TSLP treatment (FIG. 5J). Littermate controls exhibited WIHG 17 days after wounding (FIG. 5J). In contrast, mutant skin wounds did not show WIHG until 23 days after TSLP treatment (FIG. 5J). In agreement with the hair growth phenotype, cyclin D1, a key cell cycle regulator, was increased in control wounds or non-wounded tissues treated with TSLP; deletion of Tslpr in HFSC markedly diminished cyclin D1 expression in TSLP-treated wounds. Together, these data underscore that TSLP acts through TSLPR in HFSC and TAC to promote WIHG and homeostatic hair growth in the absence of wound injury.

TSLP promotes accumulation of keratinocyte progenitor factor DDX6. After wounding, HFSC and epidermal SC are activated to expand cell populations and migrate to regenerate skin appendages and fill the wound gaps. To determine additional molecular mechanisms mediating TSLP signaling in wound beds, a bioinformatics approach was used to identify differentially expressed genes (DEGs) that were upregulated in mouse skin treated with 3 µg s.c. TSLP continuously for 7 days using osmotic pumps. Those genes were cross-referenced with upregulated genes from small biopsy-induced normal healing wounds inflicted in mouse or human (Table 1). Among the top shared DEGS was the upregulation of DDX6, which encodes an RNA helicase. Notably, DDX6 maintains epidermal stem and progenitor cell identities by suppressing translation of transcripts associated with keratinocyte differentiation programs such as KLF4 .

Upregualted genes collected during transition from inflammatory to proliferative phase of healing wounds in mouse or human skin that overlap with genes upregulated following subcutaneous TSLP treatment.

TABLE 1

Gene expression analysis comparing healing skin wounds to TSLP-treated skin

| | | |
|---|---|---|
| Compare to mouse skin 24h wound vs control (GSE23006) | Compare to human day6 vs day1 wound (control) (GSE97615) | Overlapping Mouse and Human Genes |
| Ccl5 | CSF3 | DDX6 |
| Csf3 | IL1rl1 | STAT1 |
| Il1rl1 | CXCL 11 | CXCL5 |
| Cxcl11 | GRB14 | TLR2 |
| Gdap10 | IL6 | PTGS2 |
| Saa3 | CXCL2 | CD274 |
| Il6 | FABP4 | MMP3 |
| Ligp1 | MMP3 | CXCL2 |
| Cxcl2 | TNFRSF9 | IL6 |
| Gbp2 | CXCL11 | CXCL11 |
| Gbp2b | CD274 | IL1RL1 |
| Mmp3 | PTGS2 | CSF3 |
| Clec4d | TLR2 | |
| Cxcl1 | STEAP1 | |
| Cd274 | GSMB | |
| Ddx6 | SLAMF7 | |
| Cxcl1 | CTLA4 | |
| Car4 | CXCL5 | |
| Irgm2 | CXCL17 | |
| Ptgs2 | CXCL9 | |
| Tlr2 | STAT1 | |
| Gbp3 | GBP6 | |
| Ifit2 | DDX6 | |
| Cxcl5 | | |
| Stat1 | | |

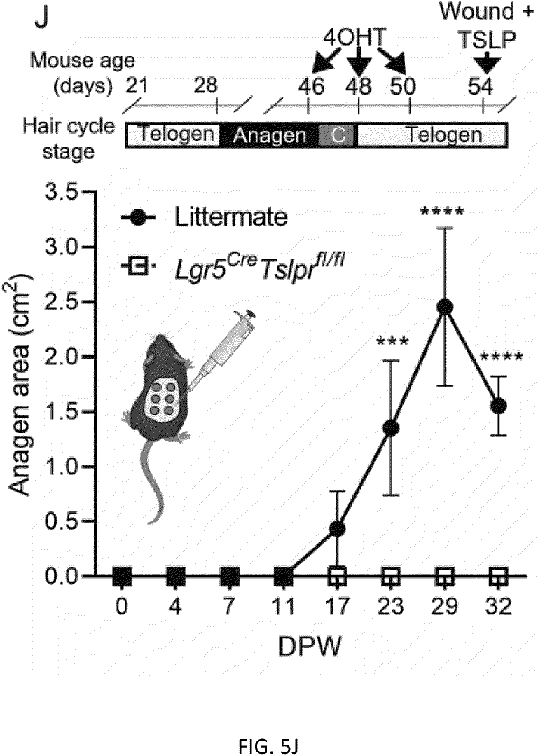

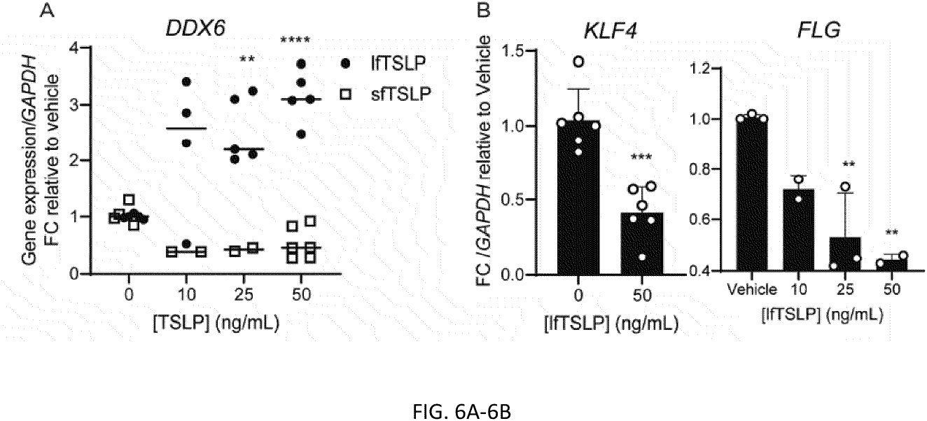

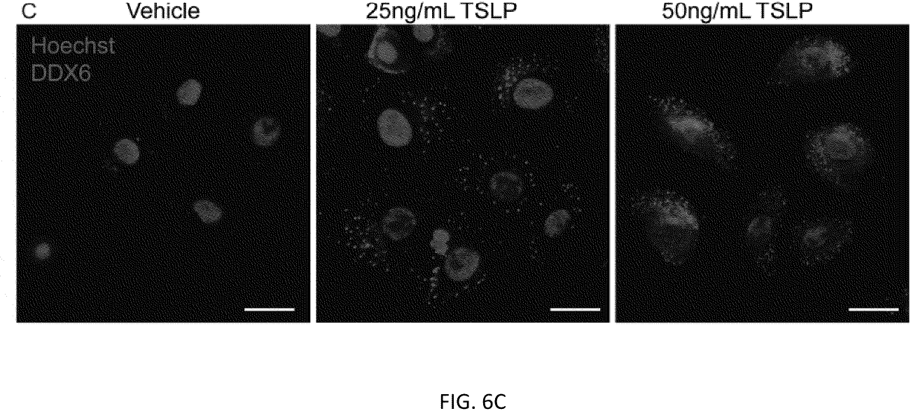

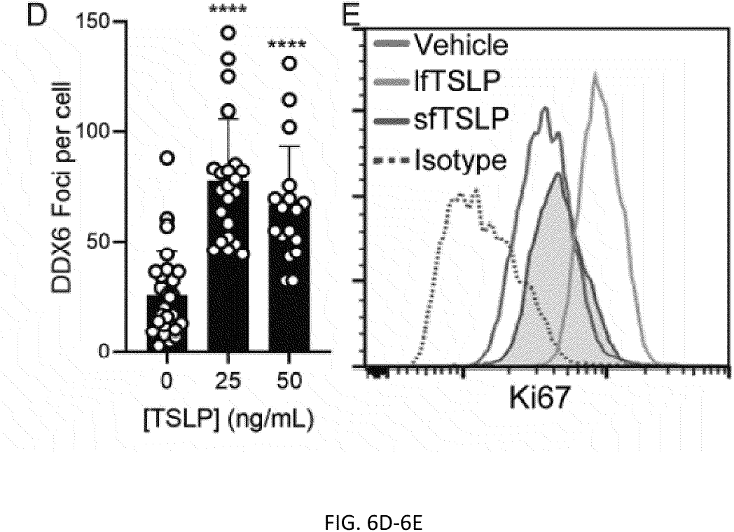

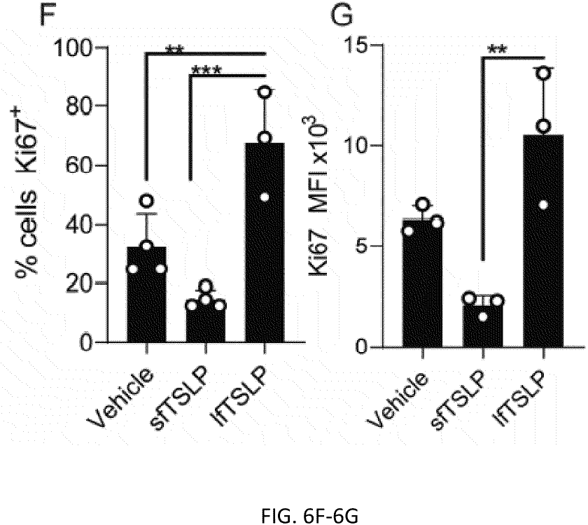

It was next evaluated whether lfTSLP has a direct role on DDX6 transcription in human epidermal keratinocytes. qPCR was used to measure transcriptional levels of DDX6 in primary normal human epidermal keratinocytes (NHEK) treated with the recombinant human lfTSLP and sfTSLP. It was found that lfTSLP treatment significantly increased expression of DDX6 by three-fold, whereas sfTSLP did not (FIG. 6A). Consistently, KLF4 and Filaggrin (FLG), both of which are differentiation markers subject to downregulation by DDX6, were decreased in cells treated by lfTSLP (FIG. 6B). Further, it was confirmed that DDX6 was detectable at the protein level via immunofluorescence staining and increased formation of distinct DDX6 foci was observed in response to lfTSLP (FIGS. 6C-D). To verify that TSLP directly alters proliferation of epidermal keratinocytes, phenotypic changes of cell proliferation marker Ki67 was assessed in epidermal keratinocytes following TSLP treatment. By flow cytometry, it was found that treatment with lfTSLP but not sfTSLP resulted in keratinocyte proliferation, as evidenced by the increases in abundance and mean fluorescence intensity (MFI) of Ki67 (FIGS. 6E-G). These data indicate that lfTSLP plays a dominant role in preserving keratinocyte stemness and promoting keratinocyte proliferation.

### Discussion

The studies provided herein demonstrate that TSLP is sufficient to initiate hair cycle activation in quiescent hair follicles and accelerate hair growth after wounding. It is reported herein that TSLP is upregulated during the anagen phase of the hair cycle and following skin injury, and peaks around 4 days after wounding. A novel function for TSLP acting on LGR5+ cells and/or their progeny to promote generation of new hair follicles following skin injury was defined. Lgr5CreER-mediated genetic deletion of TSLPR or antibody-mediated biological blockade of TSLPR prior to wounding resulted in significantly delayed hair growth. It was shown that TSLP drove generation of the TAC compartment in vivo and promoted keratinocyte proliferation in vitro. To the knowledge of the inventors, a mechanism of TSLP regulation of hair growth by acting directly on hair follicle keratinocytes has not been described previously.

TSLP is expressed by various immune cells including dendritic cells, mast cells, macrophages, eosinophils, and T cells. The data presented herein (e.g. immunostaining) suggests that keratinocytes are not the sole source of TSLP during homeostatic hair cycle and wounding healing and that additional immune and non-immune cells in the dermis contribute to local TSLP production.

It is reported that TSLP is negatively regulated by VDR and RXR signaling pathways and loss of RXR in epidermal cells results in type-2 skin inflammation in mice. K14-driven overexpression of TSLP in embryonic epidermal cells leads to skin inflammation in adult mice. Surprisingly, one dose of 100 ng or 250 ng TSLP into wounded or naïve telogen skin was neither associated with inflammatory skin erosions nor delayed wound healing. Animals treated with this amount of TSLP did not exhibit changes in scratching behavior compared to vehicle treated animals (data not shown). In contrast to the findings provided herein, s.c. injection of TSLP (2.5 µg, 10X higher than the dose used herein) into the cheek induced robust scratching behavior. Thus, the observed phenotypic differences between this study and other reports are likely due to differences of dosage, timing, and location of TSLP delivery. Without wishing to be bound by theory, it is possible that a short period of exposure of low dose TSLP is beneficial under specific circumstances such as hair growth, whereas prolonged exposure of high level TSLP induces skin and systemic atopic-like symptoms.

Recent studies have defined cues for hair cycle regulation that require heterologous cell populations and intricate crosstalk between stem cells and their progeny. TACs arise from the hair germ stem cells in response to cues from the underlying dermal papilla, which then confer signals to the bulge stem cells to complete hair growth. It was found that TSLP treatment drove the expansion of CD34+ITGα6lo cells, and this was correlated with keratinocyte TSLPR-dependent upregulation of cyclin D1.

CD34+ITGα6lo and CD34+ITGα6hi cells are defined to retain multipotent function with capacity to regenerate epidermis, hair follicle, and sebaceous glands in mice. Interestingly, CD34+ITGα6hiCD200+ cells are lost in AGA patients, whereas K15hiITGα6hi HFSC are retained in the hair bulge, but they lack growth cues in AGA and therefore unable to generate hair. TSLP was also found markedly reduced in patients with alopecia areata, and treatment with diphenylcyclopropenone elevated TSLP in patients that showed signs of hair growth. Microneedle treatments induce microinjuries to activate hair cycling and constitute a common treatment of non-scarring alopecia types, achieving an impressive efficacy of over 80%. The study provided herein provides functional insights for how wound-derived factors might drive hair growth.

In summary, this study demonstrates that TSLP promotes proliferation of HFSCs, driving wound-induced hair growth in mice.

### Example 2

Hair cycles are dependent on controlled activity of cutaneous immune cells; likewise, hair follicles are epicenters of cutaneous immune activity in the skin and participate in niche communication networks to direct the distribution and function of immune cells. However, it remains unclear whether and how HFSCs and lymphocytes communicate to resolve inflammation, promote regeneration after injury, and support hair follicle regeneration.

Hair follicles are protected by relative immune privilege mechanisms during normal physiological conditions. Local production of immunoinhibitory molecules including transforming growth factor (TGF)-β1 and IL-10 leads to the downregulation of major histocompatiblity complex (MHC) class-I on follicular keratinocytes, which depletes antigen presentation and functionally insulates the follicle from lymphocyte surveillance. The breakdown of immune privilege enables such surveillance of potential autoantigens by pre-existing, autoreactive CD8+ T cells surrounding the follicle bulb. The breakdown of immune privilege precludes the onset of alopecia areata, enabling the accumulation of CD8+ T cells.

Tissue regeneration is a remarkable process that requires cooperation of a variety of cell types to achieve homeostasis following traumatic injury. It remains unclear how HFSCs direct local immune cell distribution and function in the skin under homeostatic conditions. In this study, it is shown that HFSC TSLPR promotes postnatal hair growth (as shown in Example 1) but also plays a major role in regulation of immune cells in the dermis around the hair follicles.

### Results

TSLPR on hair follicle stem cells promotes postnatal hair growth and maintenance of normal epidermal thickness. Mammalian hair morphogenesis is tightly regulated and often regenerates in a cyclic fashion. In mouse skin, all follicles enter peak anagen by postnatal day 9, and synchronously enter catagen by postnatal day 16-17. Tslp expression was profiled using RT-qPCR during HF morphogenesis to determine whether expression patterns correlate with postnatal HF development. TSLP showed a modest decrease from early morphogenesis (postnatal day 1-2) compared to the first postnatal catagen (postnatal days 13-17, and increased considerably in the succeeding anagen cycle.

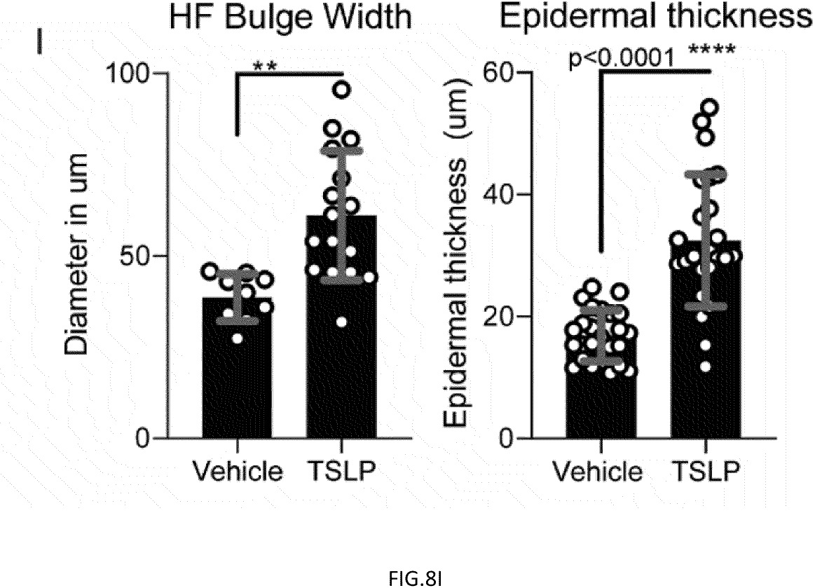

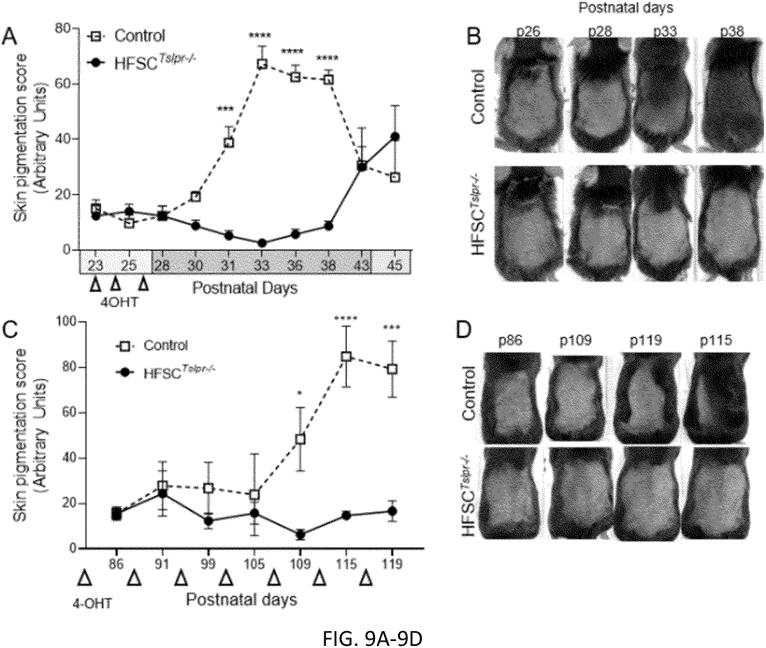

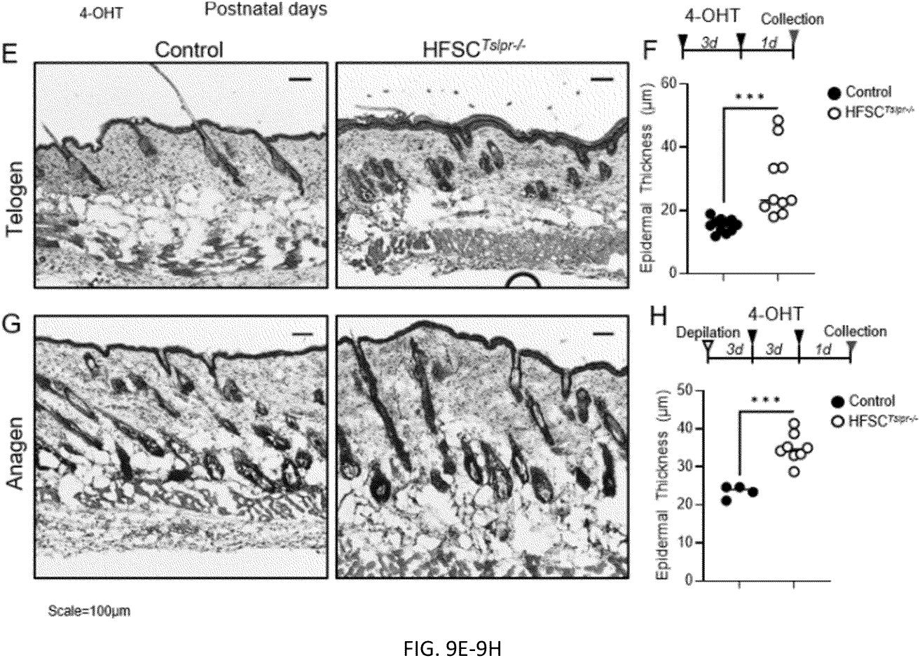

To test whether HFSC-TSLPR is required during postnatal hair development, 4-OHT was applied consecutively for 3 days to the back skin of Lgr5^{CreER} Tslpr^{fl/fl} mice and co-housed with Tslpr^{fl/fl} or Lgr5^{CreER}Tslpr^{f/+} littermates preceding the second postnatal anagen cycle. Temporally consistent anagen entry was observed in control mice treated with 4-OHT or ethanol (vehicle) by postnatal day 30 and full hair cycling by postnatal day 33-38 (FIGS. 9A-B). Strikingly, Lgr5^{CreER}Tslpr^{fl/fl} mice did not show any sign of anagen entry until postnatal day 43-nearly two weeks after their littermate controls did (FIGS. 9A-B). While anagen cycles after the second cycle are not synchronized, C57BL/6 mice exhibit episodic bouts of anagen on the back skin (CITE). To determine whether HFSC-Tslpr was required for hair cycles later in life, animals were treated with 4-OHT at 7-weeks of age and followed out for hair growth. Consistently, Lgr5^{CreER} Tslpr^{fl/fl} mice failed to enter hair cycles compared to age-matched control animals (FIGS. 9C-D). Epidermal thickening was evident following 4-OHT treatment in Lgr5^{CreER} Tslpr^{fl/fl} mice treated in both telogen (FIG. 9E-FIG. 9F) and anagen (FIGS. 9G-H). These results indicate TSLPR is required for postnatal hair cycling.

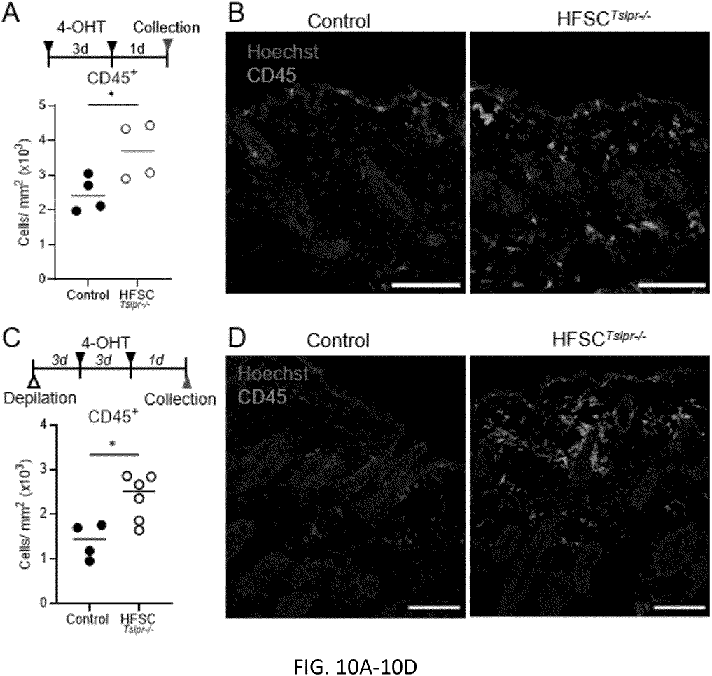

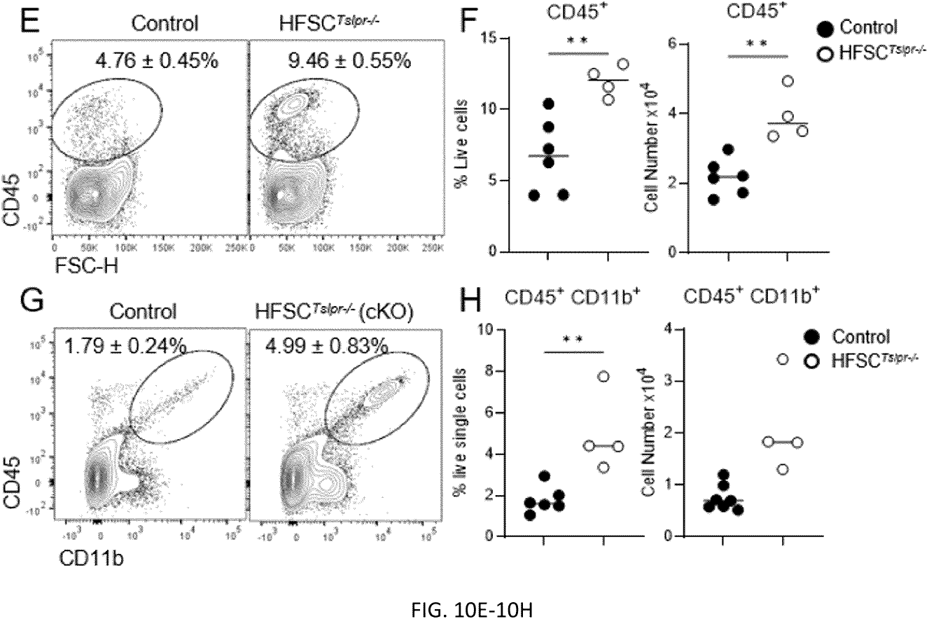

Tslpr loss on HFSC results in increased immune activity in the skin. Little is understood how HFSC TSLP signaling contributes to immune regulation. To address this, 4-OHT was administered to Lgr5^{CreER} Tslpr^{fl/fl} and Lgr5^{CreER} Tslpr^{fl/f+} mice a few days after the second synchronized anagen and immunofluorescence microscopy and flow cytometry were conducted to quantify CD45⁺ Immune cells in the skin. HFSC-specific Tslpr ablation resulted in immune cell infiltration to the tissue following Tslpr ablation in telogen (FIGS. 10A-B) and during anagen (FIGS. 10C-F).

### Tslpr Ablation During Anagen Results in Persistent Accumulation of CD8+ Immune Cells

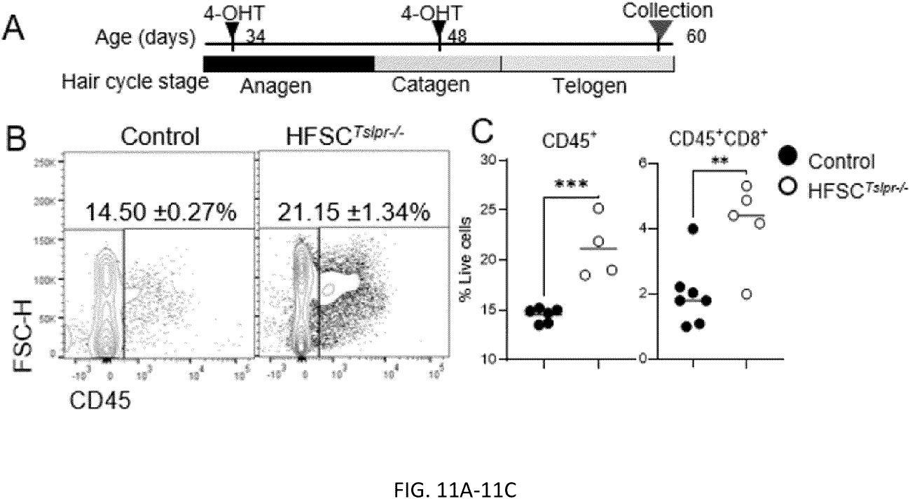

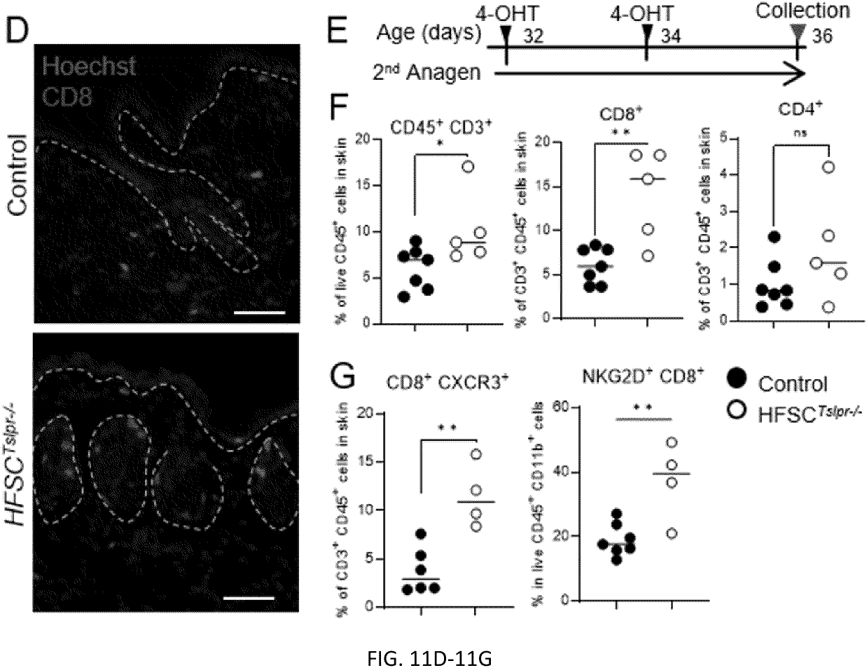

To determine whether Tslpr ablation during anagen results in persistent accumulation of immune cells in the skin, skin in anagen was treated with two doses of 4-OHT and the immune compartment was examined 26 days later (FIG. 11A). Lgr5^{CreER} Tslpr^{fl/fl} mice had significantly more CD45⁺ cells in the skin compared to their Lgr5^{CreER} Tslpr^{fl/+} littermate controls with a twofold increase in the frequency of CD8⁺ cells compared to controls (FIGS. 11B-C).

T cell frequency and spatial distribution following Tslpr ablation was observed using immunofluorescence microscopy and flow cytometry. CD8⁺ cells were located predominantly near the dermal-epidermal junction in the dermis with preferential location near hair follicles in control animals (FIG. 11D, top). Interestingly, analysis of skin sections from Lgr5^{CreER} Tslpr^{fl/lf} and Lgr5^{CreER} Tslpr^{fl/+} mice after 4-OHT treatment revealed irregular hair follicle morphology in Lgr5^{CreER} Tslpr^{fl/lf} compared to controls (FIG. 11D, bottom). More CD8⁺ cells were observed in the Lgr5^{CreER} Tslpr^{fl/fl} compared to skin from littermate controls, which also exhibited more CD8⁺ cells appearing in the epidermal layer. Dermal CD8⁺ cells still populated the space surrounding the follicle but occupied a larger range of space from the hair bulge compared to control animals. Cutaneous CD8+ T cells, but not CD4+ T cells were elevated in Lgr5^{CreER} Tslpr^{fl/lfl} mice (FIGS. 11E-3F). Thus, loss of Tslpr expression on LGR5⁺ HFSC cells appears to specifically increase CD8⁺ T cell presence in the skin.

Cytotoxic CD8⁺ T cells expressing CXCR3 and NKG2D represent dominant effectors of disease pathogenesis of alopecia areata (AA). Since Tslpr ablation delayed the onset of anagen cycles and resulted in CD8+ cell infiltration to intrafollicular space (FIG. 3D), we characterized the CD8+ T cells in Lgr5^{CreER} Tslpr^{fl/lf} mice for expression of CXCR3 and NKG2D, molecules associated with T cell migration and trafficking. By flow cytometry, it was observed that CD8+ T cells from Lgr5^{CreER} Tslpr^{fl/lf} mice expressed significantly elevated levels of CXCR3 and NKG2D compared to respective counterparts of control mice (FIG. 11G). These results indicate that TSLPR acts in HFSC to mitigate recruitment and retention of CXCR3+ NKG2D+ CD8+ T cells.

### Low Dose TSLP That Enables Hair Growth Does Not Significantly Alter Cutaneous T Cell Dynamics

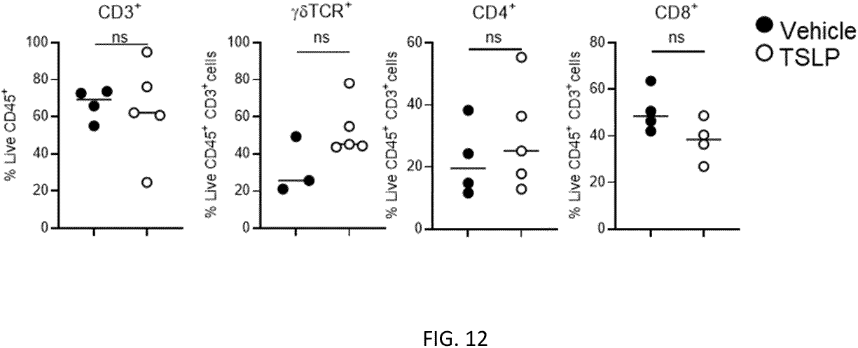

To further determine whether stem cell responses to TSLP influence lymphocyte dynamics in the skin, T cell distribution in the skin 7 days after subcutaneous TSLP injection was observed. No changes in the total frequency of CD3⁺ T cells in the skin were detected, and there was not a significant difference in cell frequency of CD4⁺, CD8⁺ or γδ T cells (CD45⁺ CD3⁺) by flow cytometry (FIG. 12). Examination of CD3⁺ or CD8⁺ by fluorescent microscopy indicated that there were no apparent changes in CD3⁺ cell frequency, but that there may be fewer perifollicular CD8⁺ cells (FIG. 12). Thus, a single s.c. TSLP injection that triggered anagen progression, did not significantly perturb T cell dynamics during the period of examination. Together, these results suggest that HFSC TSLPR signaling directs lymphocyte migration and retention in the skin.

### Example 3

### Experimental Methods

Animal studies: C57BL6/J and Lgr5CreER (the Jackson Laboratory, Bar Harbor, ME) and Tslprfl/fl mice were maintained under specific pathogen-free conditions. 4-mm or 12-mm diameter skin wounds were induced on the back of anesthetized mice during 2nd telogen unless otherwise stated. Recombinant TSLP (R&D Systems, 555-TS-010) (100 ng in 5 µL PBS) was delivered directly to wound site immediately after wound or delivered subcutaneously (250 ng in 100 uL 0.01% BSA in PBS) using a 31-gauge needle. Lgr5CreER.Tslprfl/fl mice were treated with 50 ug 4-Hydroxytamoxifen (4OHT) (Sigma # H7904) dissolved in 100% ethanol at postnatal day 46 (p46), p48, and p50. 4OHT was delivered directly to the center back skin in and wounds at p54.

Table 2 shows primers used for pPCR analyses conducted as described herein.

TABLE 2

Primers used for qPCR

| | | |
|---|---|---|
| Species and Gene | Forward (5′-3′) | Reverse (3′-5′) |
| Mouse Tslp | TTTGCCCGGAGAACAAGAG (SEQ ID NO: 1) | TTTGGACTTCTTGTGCCATTTC (SEQ ID NO: 11) |
| Human TSLPtotal | TTTGGAGATAGGCAGCTTCA G (SEQ ID NO: 2) | AGUTGTGAGGAAAGTTCAAGAG (SEQ ID NO: 12) |
| Human lfTSLP | GACTGGCAATGAGAGGCAAA (SEQ ID NO: 3) | TCTTCCCACCACGAGTGTAA (SEQ ID NO: 13) |
| Human sfTSLP | CGTAAACTTTGCCGCCTATG A (SEQ ID NO: 4) | ACTCGGTACTTTTGGTCCCACTC A (SEQ ID NO: 14) |
| Human FLG | GGTAGGTTAAGACATGAAGG ATTTGC (SEQ ID NO: 5) | GCTTGAGCCAACTTGAATACCA T (SEQ ID NO: 15) |
| Mouse Crlf2 | CTACATGACCCTGTGACCTT G (SEQ ID NO: 6) | GGCACAGGATTTGTGAGTTTG (SEQ ID NO: 16) |
| Mouse I17r | GCGTATGTCACCATGTCTAG TT (SEQ ID NO: 7) | AGCATTCCAGACTTTCCATCTC (SEQ ID NO: 17) |
| Human GAPDH | ATGGGAAGGTGAAGGTCGGA (SEQ ID NO: 8) | CAGCGTCAAAGGTGGAGGAGT5 (SEQ ID NO: 18) |
| Mouse Gapdh | GCACAGTCAAGGCCGAGAAT AGGTCGGTGTGAACGGATTT G (SEQ ID NO: 9) | GCCTTCTCCATGGTGOGTGAA TGTAGACCATGTAGTTGAGGTC A (SEQ ID NO: 19) |
| Human DDX6 | AGCCCGAGGAATCAACAATA G (SEQ ID NO: 10) | ACTGGGTAGAAAGGGAAGAGA (SEQ ID NO: 20) |

Table 3 shows primers used for genotyping analysis conducted as described herein.

TABLE 3

Primers used for Genotyping
Primers used for mouse Genotyping

| | | |
|---|---|---|
| Mouse Tslpr^{flox} | GGAGAGCAATGACGATGA GG (SEQ ID NO: 21) | GAACCCGGAAGTCATAG CAG (SEQ ID NO: 23) |
| Mouse Lgr5^{CreERT2} | CCTACTCGAAGACTTACC CAGT (SEQ ID NO: 22) | GCATTGGGGTGAATGATA GCA (SEQ ID NO: 24) |

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of promoting hair growth in a subject, comprising providing a composition containing a therapeutically effective amount of thymic stromal lymphopoietin (TSLP) to a subject in need thereof, thereby promoting hair growth in the subject.

2. The method of claim 1, wherein the composition is provided to the subject subcutaneously.

3. The method of claim 1, wherein the therapeutically effective amount of TSLP is about 0.01 µg/kg to about 20 µg/kg.

4. The method of claim 3, wherein the therapeutically effective amount of TSLP is about 0.025 µg/kg to about 10 µg/kg.

5. The method of claim 4, wherein the therapeutically effective amount of TSLP is about 0.5 µg/kg to about 5 µg/kg.

6. The method of claim 4, wherein the therapeutically effective amount of TSLP is about 1 µg/kg to about 3 µg/kg.

7. The method of claim 1, wherein the subject has experienced a skin injury.

8. The method of claim 7, wherein the composition is provided to the subject subcutaneously at an area of injured skin.

9. The method of claim 1, wherein the subject has a hair disorder.

10. The method of claim 9, wherein the hair disorder is alopecia.

11. A method of promoting hair growth in a subject, comprising providing a composition containing a therapeutically effective amount of thymic stromal lymphopoietin (TSLP) to a subject, wherein the subject has alopecia, and wherein the composition is provided to the subject following a microneedling procedure performed on the subject.

12. The method of claim 11, wherein the composition is administered subcutaneously at one or more microneedling sites.

13. The method of claim 11, wherein the therapeutically effective amount of TSLP is about 0.01 µg/kg to about 20 µg/kg.

14. The method of claim 13, wherein the therapeutically effective amount of TSLP is about 0.025 µg/kg to about 10 µg/kg.

15. The method of claim 14, wherein the therapeutically effective amount of TSLP is about 0.5 µg/kg to about 5 µg/kg.

16. The method of preventing chemotherapy-induced hair loss in a subject, the method comprising providing a composition containing a therapeutically effective amount of an inhibitor of thymic stromal lymphopoietin (TSLP) to a subject, wherein the inhibitor of TSLP is provided to the subject before, during, and/or after chemotherapy, thereby preventing chemotherapy-induced hair loss in the subject.

17. The method of claim 16, wherein the composition is provided to the subject subcutaneously.

18. The method of claim 17, wherein the inhibitor of TSLP comprises an antibody.

19. The method of claim 18, wherein the inhibitor of TSLP comprises Tezepelumab.

20. The method of claim 16, wherein the subject is a human.
